(19) United States
(12) Patent Application Publication
(10) Pub. No.: US 2025/0265742 A1
(43) Pub. Date: Aug. 21, 2025

(54) MODULAR MATERIAL DECOMPOSITION FROM ENERGY RESOLVING PHOTON COUNTING DATA

(71) Applicants: GE Precision Healthcare LLC, Waukesha, WI (US); Purdue Research Foundation, West Lafayette, IN (US)

(72) Inventors: Jonathan S. Maltz, Oakland, CA (US); Charles A. Bouman, JR., West Lafayette, IN (US); Natalie Jadue, Cuyahoga Falls, OH (US); Madhuri Mahendra Nagare, Sunnyvale, CA (US); Gregery T. Buzzard, West Lafayette, IN (US)

(21) Appl. No.: 19/051,713

(22) Filed: Feb. 12, 2025

Related U.S. Application Data
(60) Provisional application No. 63/554,266, filed on Feb. 16, 2024.

(51) Int. Cl. G06T 11/00; A61B 6/00; A61B 6/03; A61B 6/42

(52) U.S. Cl. CPC G06T 11/008; A61B 6/032; A61B 6/4241; A61B 6/5258

(57) ABSTRACT

One or more systems, devices, computer program products and/or computer-implemented methods of use provided herein relate to modular material decomposition from energy resolving photon counting data. According to an embodiment, a system is provided. The system can further comprise a processor that can execute computer-executable components stored in memory, wherein the computer-executable components can comprise a reconstruction component that can reconstruct one or more material decomposed CT images by balancing a forward model agent and a prior model agent, wherein the forward model agent can represent a conditional distribution of observed data given an unknown CT image, wherein the prior model agent can represent an assumed prior distribution, and wherein the observed data can include measurements from a PCD.

### CROSS-REFERENCE TO RELATED APPLICATION

This application claims the benefit of U.S. Provisional Application Ser. No. 63/554,266, filed Feb. 16, 2024 and entitled MODULAR MATERIAL DECOMPOSITION FROM ENERGY RESOLVING PHOTON COUNTING DATA, which is incorporated herein by reference.

### TECHNICAL FIELD

The subject disclosure relates generally to image processing and, more specifically, to modular material decomposition from energy resolving photon counting data.

### BACKGROUND

X-ray computed tomography (CT) based on photon counting detectors (PCD) extends standard CT by counting detected photons in multiple energy bins. PCD data can be used to increase a contrast-to-noise ratio (CNR), increase spatial resolution, reduce radiation dose, reduce injected contrast dose, and compute a material decomposition using a specified set of basis materials. Current commercial and prototype clinical photon counting CT systems utilize PCD-CT reconstruction methods that either reconstruct from each spectral bin separately, or first create an estimate of a material sinogram using a specified set of basis materials and then reconstruct from these material sinograms. However, existing methods cannot utilize simultaneously and in a modular fashion, both the measured spectral information and advanced prior models in order to produce a material decomposition.

Accordingly, systems or techniques for more versatile material decomposition can be desirable.

### SUMMARY

The following presents a summary to provide a basic understanding of one or more embodiments described herein. This summary is not intended to identify key or critical elements, delineate scope of particular embodiments or scope of claims. Its sole purpose is to present concepts in a simplified form as a prelude to the more detailed description that is presented later. In one or more embodiments described herein, systems, computer-implemented methods, apparatus and/or computer program products that enable modular material decomposition from energy resolving photon counting data are discussed.

According to an embodiment, a system is provided. The system can comprise a processor that can execute computer-executable components stored in memory, wherein the computer-executable components can comprise a reconstruction component that can reconstruct one or more material decomposed CT images by balancing a forward model agent and a prior model agent, wherein the forward model agent can represent a conditional distribution of observed data given an unknown CT image, wherein the prior model agent can represent an assumed prior distribution, and wherein the observed data can include measurements from a photon counting detector.

According to another embodiment, a computer-implemented method is provided. The computer-implemented method can comprise reconstructing, by a device operatively coupled to a processor, one or more material decomposed CT images by balancing a forward model agent and a prior model agent, wherein the forward model agent can represent a conditional distribution of observed data given an unknown CT image, wherein the prior model agent can represent an assumed prior distribution, and wherein the observed data can include measurements from a photon counting detector.

According to yet another embodiment, a computer program product for modular material decomposition from energy resolving photon counting data is provided. The computer program product can comprise a non-transitory computer readable memory having program instructions embodied therewith, the program instructions executable by a processor to cause the processor to reconstruct one or more material decomposed CT images by balancing a forward model agent and a prior model agent, wherein the forward model agent can represent a conditional distribution of observed data given an unknown CT image, wherein the prior model agent can represent an assumed prior distribution, and wherein the observed data can include measurements from a photon counting detector.

According to yet another embodiment, a system is provided. The system can comprise a processor that can execute computer-executable components stored in memory, wherein the computer-executable components can comprise a reconstruction component that can reconstruct one or more material decomposed CT images by iteratively applying a forward model agent and a prior model agent, wherein reconstructing the one or more material decomposed CT images can involve using photon counting measurements from a photon counting detector, wherein the forward model agent can minimize a negative log likelihood of the photon counting measurements, and wherein the prior model agent can represent a second function that is an assumed prior distribution.

According to yet another embodiment, a system is provided. The system can comprise a processor that can execute computer-executable components stored in memory, wherein the computer-executable components can comprise a reconstruction component that can reconstruct one or more material decomposed CT images by iteratively balancing a plurality of functions, wherein reconstructing the one or more material decomposed CT images can involve using photon counting measurements from a photon counting detector, wherein at least a first function of the plurality of functions can be a forward model agent that can minimize a fit of measured photon counts, and wherein at least a second function of the plurality of functions can be a prior model agent that can reduce noise in one or more reconstructed material decomposed CT images.

### BRIEF DESCRIPTION OF THE DRAWINGS

One or more embodiments are described below in the Detailed Description section with reference to the following drawings:

FIG. 1 illustrates a block diagram of an example, non-limiting system that can perform modular material decomposition from energy resolving photon counting data in accordance with one or more embodiments described herein.

FIG. 2 illustrates example, non-limiting graphs of material space sample locations used to calibrate a detector response function (DRF) in accordance with one or more embodiments described herein.

FIG. 3 illustrates a flow diagram of an example, non-limiting method that can perform modular material decomposition from energy resolving photon counting data in accordance with one or more embodiments described herein.

FIG. 4 illustrates example, non-limiting reconstructions formed as a linear combination of material decomposed reconstructions weighted to simulate a 70 kiloelectron volt (keV) monoenergetic scan in accordance with one or more embodiments described herein.

FIG. 5 illustrates an example, non-limiting reconstruction of a small gammex in accordance with one or more embodiments described herein.

FIG. 6 illustrates an example, non-limiting set of MLE and MACE reconstructions for measured data with a small gammex phantom in accordance with one or more embodiments described herein.

FIG. 7 illustrates example, non-limiting MACE reconstructions for measured data with a small gammex phantom and a lungman phantom in accordance with one or more embodiments described herein.

FIG. 8 illustrates a flow diagram of an example, non-limiting method that can perform modular material decomposition from energy resolving photon counting data in accordance with one or more embodiments described herein.

FIG. 9 illustrates an example reconstruction with bad detectors.

FIG. 10 illustrates an example of a poor detector creating rings in reconstruction.

FIG. 11 illustrates an example of reducing bias in a sinogram.

FIG. 12 illustrates a block diagram of an example, non-limiting operating environment in which one or more embodiments described herein can be facilitated.

FIG. 13 illustrates an example networking environment operable to execute various implementations described herein.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

CT based on PCDs extends standard CT by using measurements of photons at multiple energies. When coupled with the spatial content inherent in CT, these spectral measurements have the potential to yield clinically relevant improvements to CT images, including increased CNR, increased spatial resolution, reduced radiation and contrast dose, and material decomposition using a specified set of basis materials. Some typical use cases for such high-quality reconstructions include applications involving high spatial resolution and high CNR, such as identification of small low-contrast lesions in thin slices, imaging breast tissue, temporal bones, chests, hearts, etc. Realizing the full potential of photon counting CT (PCCT) for increased spatial resolution without increasing radiation dose can involve the use of algorithmic methods to boost CNR.

Methods for image reconstruction from PCD data fall largely into two classes, wherein a first class includes creating one reconstruction for each spectral bin, and wherein a second class includes creating a material decomposition in a specified material basis, either directly in image space or first in sinogram space followed by a reconstruction. Reconstructing each spectral bin is straightforward and can be achieved using filtered backprojection or with iterative methods, but the bin-wise approach limits the ability to correct for beam hardening and to exploit the mutual information between bins. On the contrary, a material decomposition can produce one image for each of a set of basis materials, typically two primary materials. It is to be appreciated that for K-edge imaging with multiple contrast agents, more material basis functions can be utilized. A material decomposition in image space can map bin-wise reconstructions to material images, but this approach has a limited ability to correct for beam-hardening. Existing methods to reduce noise in material pathlength sinograms resulting from material decomposition in sinogram or projection space are not able to utilize simultaneously and in a modular fashion, both the measured spectral information and advanced prior models in order to produce a material decomposition. Existing techniques for CT image reconstructions are also not suitable for use with a Deep Silicon (Si) detector. Thus, efficient techniques for PCD-based CT reconstructions and material decompositions can be desirable.

Various embodiments of the present disclosure can be implemented to produce a solution to one or more of the problems discussed above. Embodiments described herein include systems, computer-implemented methods, and computer program products that can enable a modular framework for PCD-based CT reconstruction and material decomposition based on a Multi-Agent Consensus Equilibrium (MACE) framework. A material decomposition can produce one image for each of a set of basis materials, typically two primary materials. In various embodiments, for material decomposition in sinogram or projection space, the photon counts across energy bins at a single projection location can be mapped to an estimate of pathlength integrals of the material basis components. Such a technique can employ a maximum likelihood estimator (MLE) or an approximation to the MLE to model a Poisson distribution of photon counts. Further, various embodiments herein can utilize a detector proximal map (DPM) or agent that can use PCD measurements to update an estimate of the pathlength sinogram. In various embodiments, a prior agent in the form of a sinogram denoiser that can enforce both physical and empirical knowledge about a material-decomposed sinogram can also be created. A sinogram reconstruction can be computed using the MACE algorithm, wherein the MACE algorithm can compute an equilibrium solution between the two agents, and a final image can be reconstructed from the estimated sinogram by another algorithm. A modularity of the techniques disclosed herein can allow the two agents (i.e., the DPM agent and the prior agent) to be designed, implemented, and optimized independently. Experiments conducted with simulated and measured data showed that the proposed MACE-based reconstruction algorithm can outperform the MLE. On a low-contrast test phantom, the CNR of the techniques disclosed herein was observed to be 4.5 times the CNR of the MLE. That is, experimental results generated from the techniques disclosed herein showed a substantial (450%) CNR boost in comparison to a conventional maximum likelihood reconstruction when applied to a phantom used to evaluate low contrast detectability.

Embodiments of the present disclosure can provide several technical and commercial advantages that can be achieved with the MACE algorithm. For example, the techniques disclosed herein can provide an opportunity to reduce dosage during CT scanning by offering extensive noise reductions with minimal reduction in resolution, and the techniques disclosed herein can be employed directly with current data processing chains for a photon counting CT scanner. Additionally, the forward model and calibration account for effects due to Compton scatter in detector channels, a bowtie filter and the non-uniform detector design. The framework developed herein can be modular, which can allow individual components in algorithms to be modelled with advanced methods. Furthermore, the techniques disclosed herein have a CNR 4.5 times better than the MLE solution, as stated elsewhere herein.

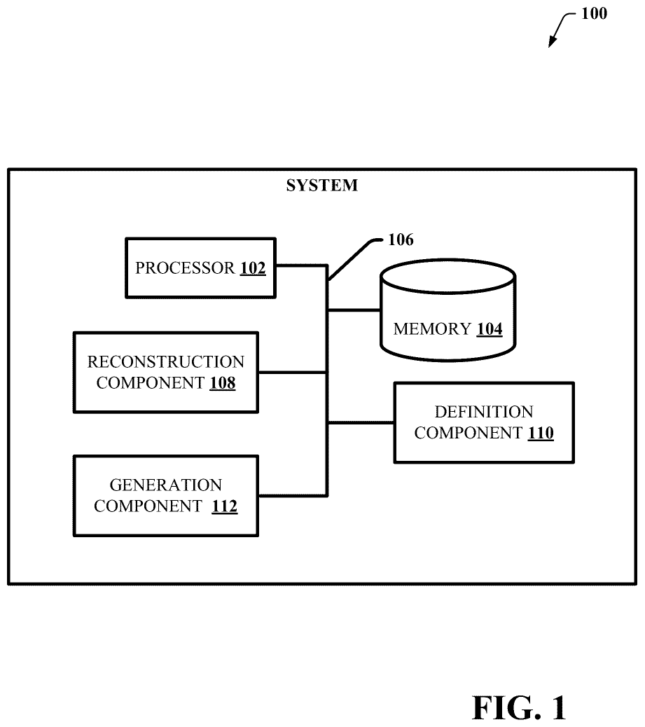

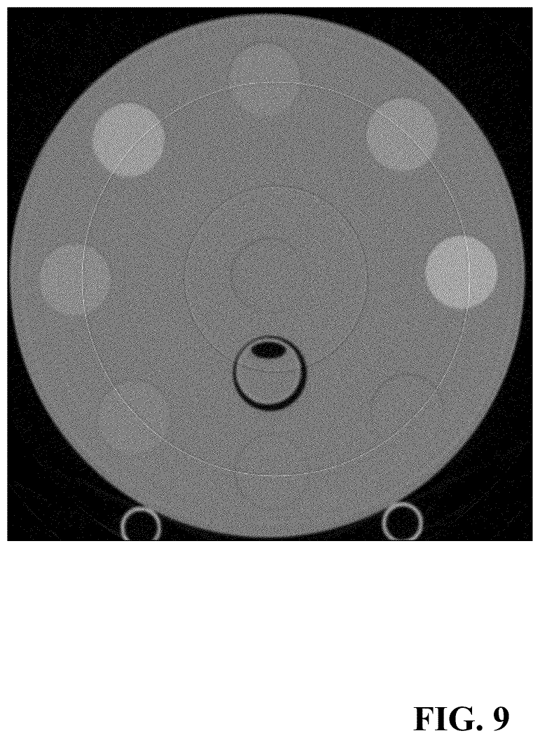

The embodiments depicted in one or more figures described herein are for illustration only, and as such, the architecture of embodiments is not limited to the systems, devices and/or components depicted therein, nor to any particular order, connection and/or coupling of systems, devices and/or components depicted therein. For example, in one or more embodiments, the non-limiting systems described herein, such as non-limiting system 100 as illustrated at FIG. 1, and/or systems thereof, can further comprise, be associated with and/or be coupled to one or more computer and/or computing-based elements described herein with reference to an operating environment, such as the operating environment 900 illustrated at FIG. 9. For example, system 100 can be associated with, such as accessible via, computing environment 900 described below with reference to FIG. 9, such that aspects of processing can be distributed between system 100 and the computing environment 900. In one or more described embodiments, computer and/or computing-based elements can be used in connection with implementing one or more of the systems, devices, components and/or computer-implemented operations shown and/or described in connection with FIG. 1 and/or with other figures described herein.

FIG. 1 illustrates a block diagram of an example, non-limiting system 100 that can perform modular material decomposition from energy resolving photon counting data in accordance with one or more embodiments described herein.

System 100 and/or the components of system 100 can be employed to use hardware and/or software to solve problems that are highly technical in nature (e.g., related to PCD-based CT imaging, CT image reconstruction, material decomposition, etc.), that are not abstract and that cannot be performed as a set of mental acts by a human. Further, some of the processes performed may be performed by specialized computers for carrying out defined tasks related to modular material decomposition from energy resolving photon counting data. The system 100 and/or components of the system can be employed to solve new problems that arise through advancements in technologies mentioned above, and/or the like. System 100 can provide technical improvements to CT image reconstruction systems by reducing noise in material-basis and virtual monoenergetic images. More specifically, PCCT can achieve high spatial resolution without a dose penalty by allowing efficient imaging using small pixels. PCCT (photon counting CT) detector pixels are generally smaller than EID (energy integrating detector) pixels. For example, in clinical CT scanners: typical energy-integrating detector cells/pixels are 1 mm (although there are some exceptions around 0.5 mm and relatively older scanners have larger cells around 2 mm). Typical photon-counting detector cells/pixels are 0.4-0.7 mm (but other cell sizes can being considered).

At a given dose, this can imply that each pixel receives fewer photon counts than the larger pixels of standard CT detectors. When counts per pixel approach zero, obtaining spectral resolution can become difficult or impossible. In this regard, embodiments of the present disclosure can use regularization to overcome the problem of obtaining spectral resolution, and greatly reduce noise in material-basis and virtual monoenergetic images. Embodiments of the present disclosure can also employ a photon counting strategy based on offering the best spatial and spectral resolution. Additionally, system 100 can assist patients and healthcare professionals to realize the benefits of PCCT without escalating dosage, and provide a more accurate material differentiation, which can be particularly beneficial for large patients/anatomies.

Discussion turns briefly to processor 102, memory 104 and bus 106 of system 100. For example, in one or more embodiments, system 100 can comprise processor 102 (e.g., computer processing unit, microprocessor, classical processor, and/or like processor). In one or more embodiments, a component associated with system 100, as described herein with or without reference to the one or more figures of the one or more embodiments, can comprise one or more computer and/or machine readable, writable and/or executable components and/or instructions that can be executed by processor 102 to enable performance of one or more processes defined by such component(s) and/or instruction(s).

In one or more embodiments, system 100 can comprise a computer-readable memory (e.g., memory 104) that can be operably connected to processor 102. Memory 104 can store computer-executable instructions that, upon execution by processor 102, can cause processor 102 and/or one or more other components of system 100 (e.g., reconstruction component 108, definition component 110, and/or generation component 112) to perform one or more actions. In one or more embodiments, memory 104 can store computer-executable components (e.g., reconstruction component 108, definition component 110, and/or generation component 112).

System 100 and/or a component thereof as described herein, can be communicatively, electrically, operatively, optically and/or otherwise coupled to one another via bus 106. Bus 106 can comprise one or more of a memory bus, memory controller, peripheral bus, external bus, local bus, and/or another type of bus that can employ one or more bus architectures. One or more of these examples of bus 106 can be employed. In one or more embodiments, system 100 can be coupled (e.g., communicatively, electrically, operatively, optically and/or like function) to one or more external systems (e.g., a non-illustrated electrical output production system, one or more output targets, an output target controller and/or the like), sources and/or devices (e.g., classical computing devices, communication devices and/or like devices), such as via a network. In one or more embodiments, one or more of the components of system 100 can reside in the cloud, and/or can reside locally in a local computing environment (e.g., at a specified location(s)).

As described above, system 100 can comprise one or more computer and/or machine readable, writable and/or executable components and/or instructions that, when executed by processor 102, can enable performance of one or more operations defined by such component(s) and/or instruction(s). For example, in various embodiments, reconstruction component 108 can reconstruct one or more material decomposed CT images by balancing two update maps, a first update map being a forward model agent and a second update map being a prior model agent, wherein the forward model agent can represent a conditional distribution of observed data given an unknown CT image, wherein the prior model agent can represent an assumed prior distribution, and wherein the observed data can include measurements from a PCD. In various embodiments, the forward model agent and the prior model agent can be generated by generation component 112, and generating the forward model agent and prior model agent can decompose a CT image reconstruction problem into separate algorithms (i.e., the forward model agent and the prior model agent) that can be tuned individually. In various embodiments, the prior model agent can be designed/trained using training data and machine learning techniques for generating a smooth reconstructed CT image.

More specifically, in a Bayesian formulation, the reconstruction problem discussed herein can refer to estimating an unknown image x from observed photon counts y using an assumed prior distribution, P(x). Letting y∈ [symbol] ^{M×K} be the measurements from a PCD with M projections and K energy or spectral bins, reconstruction component 108 can aim to reconstruct x∈ [symbol] ^{N×L} representing a fractional volume of L basis materials in each of the reconstructed N voxels, from y. Then, a maximum a posteriori (MAP) estimate for x can be given by Equation 1.

$${\hat{x}}_{MAP} = {arg min}_{x ∈ Ω} \{ - log P ( y \vert x ) - log P ( x ) \} , \qquad Equation 1$$

where Ω represents the set of possible values for x, P(y|x) represents the conditional distribution of the observed data y given the unknown image x (or the forward model relating x to y), and P(x) represents the prior model.

In various embodiments, definition component 110 can define the forward model agent and the prior model agent in a path length sinogram domain. The forward model agent can be designed to reduce a negative log likelihood of the measurements from the photon counting device. Additionally, the forward model agent can be designed to estimate path lengths of basis materials, wherein the forward model agent can reduce a negative log likelihood of a Poisson photo counting model, and wherein a DRF in the forward model agent can be approximated by a low-degree polynomial or a member of a low-dimensional set of functions. Basis materials can often be water and bone, or water and iodine, polyethylene (PE) and polyvinyl chloride (PVC), etc. The basis materials can be selected based on the two different ways in which a photon can interact with any material. For example, in X-ray technology, a photon can interact with an object in two primary ways, namely, the photoelectric effect and the Compton effect. As a result, a material can be identified as a ratio between the two effects based on other properties of the original object that can be estimated.

More specifically, an intermediate pathlength sinogram linking y to x can be incorporated to prompte modularity. For example, in various embodiments, A∈ [symbol] ^{M×N} can denote a system matrix in distance units (centimeters (cm)) so that the pathlengths of each basis material through the phantom can be given by p=Ax. Herein, A_{m,n} represents the length of intersection of a projection ray, m, with voxel, n, and p_{m,l} represents a distance in cm that the projection ray m can travel through the basis material l. Using p=Ax in Equation 1, the forward model, P(y|x), can be reformulated as a detector forward model, P_{d}(y|p), that can capture details of the detector design by relating the measurements, y, to the pathlength sinogram, p. By defining Ωₚ as the set of possible values of p and assuming a reconstruction algorithm that can reconstruct x from p, a sinogram-based reconstruction approach given by Equation 2 can be obtained.

$${\hat{p}}_{MAP} = {arg min}_{p ∈ {Ω}_{p}} \{ f ( p ) + h ( p ) \} , \\ where f ( p ) = - log {P}_{d} ( y \vert p ) \qquad Equation 2$$

represents the detector forward model and h(p)=−log P(p) represents the prior model in p. The prior model can regularize the estimate, while the detector forward model P_{d}(y|p) can account for various physical effects in a photon counting detector, including photon binning, source flux, and detector scatter.

The Bayesian approach presented in Equation 2 can experience challenges in terms of a need to specify prior information using a function h(p), wherein such prior information can be unavailable for advanced priors such as neural networks. To address this problem, embodiments of the present disclosure can convert the functions ƒ and h to proximal maps, followed by solving Equation 2 using reconstruction component 108. In various embodiments, reconstruction component 108 can be a MACE algorithm. The resulting algorithm can generalizes immediately to allow the use of advanced priors.

Applying the MACE algorithm can be preceded by defining the detector agent (or forward model), F(p; y), and the prior agent, H(p), according to Equation 3 and Equation 4, respectively.

$$F ( p ; y ) = {arg min}_{q} \{ f ( q ) + \frac{1}{2 {σ}^{2}} {‖ q - p ‖}^{2} \} \qquad Equation 3$$

$$H ( p ) = {arg min}_{q} \{ h ( q ) + \frac{1}{2 {σ}^{2}} {‖ q - p ‖}^{2} \} , \qquad Equation 4$$

where σ represents a parameter that can affect the convergence rate.

Based on the definitions given by Equations 3 and 4, the MAP estimate of Equation 2 can satisfy \hat{p}_{MAP}=p*, where F(p*−u*; y)=p*=H(p*+u*) according to Equation 5. Stated differently, using the definitions of Equations 3 and 4, the MAP estimate of Equation 2 can be solved by using Algorithm 1.

$$F ( {p}^{*} - {u}^{*} ; y ) = {p}^{*} = H ( {p}^{*} + {u}^{*} ) , \qquad Equation 5$$

where u* can be an estimate of noise removed by the prior agent H. Equations 3-5 are known as consensus equilibrium equations, and Equation 3-5 can be solved by using Algorithm 1. The equivalent formulation in Equation 5 can allow for the use of neural networks or other advanced priors for H(p). That is, the MACE algorithm can solve a more general class of problems in addition to solving for a MAP estimation by using different agents, H(p), wherein H(p) can be a denoising operator that can be either image-independent, parametric or trained using machine learning methods. When H(p) can be defined directly rather than as a proximal map for h(p), the MACE reconstruction may not be the solution to an optimization problem, but the MACE reconstruction can be still a solution to an equilibrium problem having the form p*=F(p*−u*)=H(p*+u*). In experiments conducted in connection with the embodiments discussed herein, H(p) was chosen to be a linear, space invariant filter with a Gaussian point spread function (psf).

### Algorithm 1: Basic MACE Algorithm

Input: y (data), pᵢₙᵢₜ (initial reconstruction), ρ (step size), H (denoiser), F (data-fitting update)
Update: p (final reconstruction in material path lengths)
   1. p←pᵢₙᵢₜ
   2. for i=0: N_{MACE}−1 do
   3. p₁←2H(p)−p
   4. p′←F(p₁; y)
   5. p₁←2p′−p₁
   6. p←(1−ρ)p+ρp₁
   7. end for
Return: p′

### Photon Counting Detector Model:

In the simplest case, a Poisson distribution of photon counts can be assumed, so that the data y given p can be a conditional Poisson random variable with mean λ(p), where λ(p)∈ [symbol] ^{M×K} are expected photon counts for known path lengths p∈ [symbol] ^{M×L}. Letting ψ and ρ be mass attenuation coefficients and material density for L basis materials, respectively, the i^{th} row (projection ray) of λ(p) can be given by λ(pᵢ)=[λ₁(pᵢ), . . . , λ_{K}(pᵢ)]=∫₀^{∞} exp(−pᵢ[ψ(e)⊙ρ]) W^{NC} (e) de, where W^{NC} (e) is a term indicating effective incident X-ray flux and detector efficiency at energy e when Compton scatter in the detector is not considered. The detector Compton scatter can also be modeled, which shifts the count distribution from higher to lower energy bins and can lead to multiple counts per primary photon. A matrix R∈ [symbol] ^{K×K} can be used to capture these effects and replace W^{NC}(e) in λ(pᵢ)=[λ₁(pᵢ), . . . , λ_{K}(pᵢ)]=∫₀^{∞} exp(−pᵢ[ψ(e)⊙ρ]) W^{NC}(e) de with W(e)=W^{NC}(e) R. Finally, to account for variations such as the use of a bowtie or nonuniform detector response, a detectorwise airscan can be used to normalize to obtain \tilde{λ}(p)=λ(p)/λ(0), which can be used to formulate the detector log likelihood.

The detector negative log likelihood in pathlength can be given by ƒ(p)=−log P(y|p), that the embodiments herein can aim to minimize. Using the Poisson assumption and dropping terms independent of p, ƒ(p) can be expressed in terms of \tilde{λ}(p). However, since raw measurements of \tilde{λ}(p) can be usually unavailable, the negative log attenuation can be defined as

$$ϕ ( p ) = - log \tilde{λ} ( p ) = - log \frac{E [ Y ]}{λ ( 0 )} .$$

Based on the definition of the negative log attenuation, ƒ(p)=(exp(−ϕ(p))∘λ(0)+ϕ(p)∘y) 1. For PCDs with relatively narrow energy bins, each spectral component in the λ(pᵢ) equation can be roughly a constant times an exponential in pᵢ. Thus, ϕ(p) can be approximately a linear function of p. To model non-linear effects such as beam hardening, and scattering, ϕ(p) can be approximated with a low-degree polynomial, ϕ_{θ}(p), that can be used in place of ϕ(p) in ƒ(p)=(exp(−ϕ(p))∘λ(0)+ϕ(p)∘y) 1.

To compute the MAP estimate, \hat{p}_{MAP}, the cost function ƒ(p)+h(p) in Equation 2 can be jointly minimized. In the case that h is given explicitly in a computationally tractable form, ADMM can be used, which relies on variable splitting, sequential application of proximal maps for ƒ and h, and a Lagrange update term. The DPM F_{d}(p) can be implemented using a quadratic surrogate function. That is, the approximate linearity of ϕ(p) can allow ϕ(p) in ƒ(p)=(exp(−ϕ(p))∘λ(0)+ϕ(p)∘y) 1 to be replaced with a linear A p, followed by using a quadratic approximation for the resulting expression. Using ƒₛ to denote the surrogate, the DPM can take the form

$${F}_{d} ( p ) = {arg min}_{q} \{ {f}_{s} ( q ) + \frac{1}{2 {σ}^{2}} {‖ q - p ‖}^{2} \} .$$

The DPM or agent can have the interpretation of taking an input estimate, p, and improving the input estimate according to the cost function, ƒₛ, while maintaining proximity to p through the quadratic distance penalty. In contrast to an explicit h(p), the prior may be encoded implicitly as a denoiser or other image enhancement algorithm, including a neural network denoiser. In this case, MACE can be used to reconcile multiple agents to achieve a consensus solution, \hat{p}_{MAP}, followed by applying 3D FDK on \hat{p}_{MAP} to reconstruct \hat{x}. The modular implementation of the DPM, F_{d}(p), can assist with the implementation of the MACE algorithm. The function, F_{d}(p), can embody details of the detector physics through the design of the function ƒ(p). In addition, F_{d}(p) can be designed to be computationally efficient. The following discussion elaborates upon the photon counting detector model in detail.

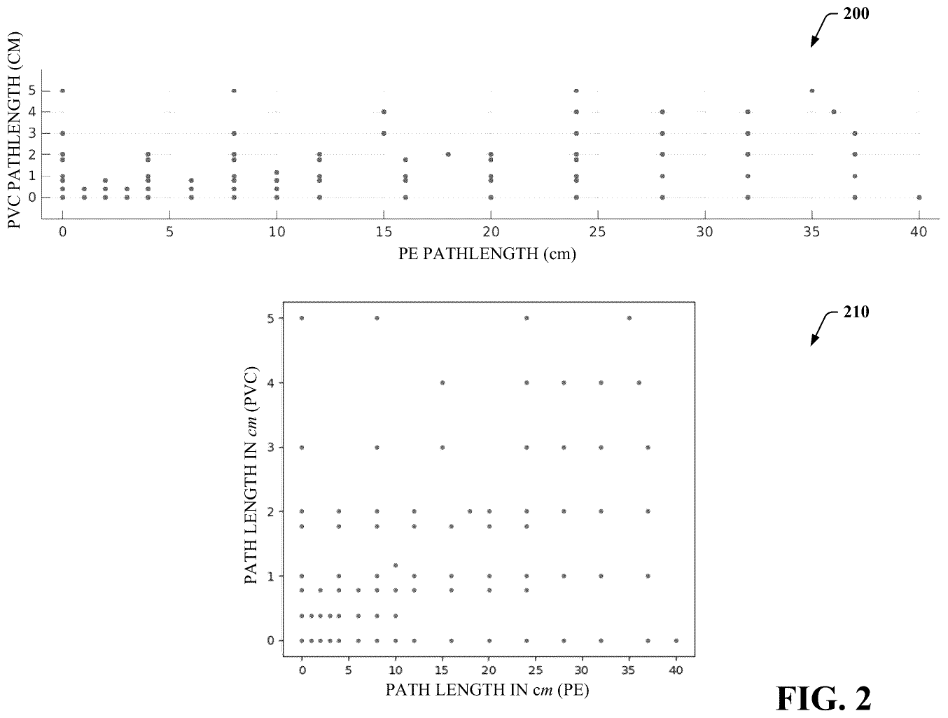

In various embodiments, a simplified model for the detector log likelihood, ƒ(p)=−log P_{d}(y|p), based on the properties of a photon counting detector can be derived. The model can be calibrated and efficiently computed using a quadratic surrogate function for ƒ that can allow for fast approximation of F(p; y), as explained in greater detail infra with reference to FIG. 2. To define ƒ, let yⱼ∈ [symbol] ^{K} be the vector of photon counts for each of K energy bins for the j^{th} projection. Assuming yⱼ to be composed of independent Poisson distributed random variables with mean λⱼ(p)∈ [symbol] ^{K} where p∈ [symbol] ^{L} is a vector of path lengths for each of L basis materials, it can be assumed that for each detector the expected number of photon counts, λⱼ(p), are completely determined by the path length through the object being imaged, and that the distribution of those photon counts are independent and identically distributed (i.i.d.) and Poisson. It is to be noted that this is an approximation since the expected photon count can vary due to effects such as scatter, and that the distribution of the counts can vary from i.i.d. and Poisson due to effects such as Compton scatter in the detector, pile-up, and other nonlinear interactions.

In practice, the raw photon counts y_{j,k}, are not given since the photon counts are typically first normalized by the total photon count. Consequently, a measurement for the j^{th} projection can be given by the vector Tⱼ=[T_{j,0}, . . . , T_{j,K-1}]∈ [symbol] ^{K}, where Tⱼ can be further given by Equation 6, and λ_{j,Σ}=Σₖ₌₁^{K} λ_{j,k} (0) represents a scalar representing the total photon count for the j^{th} projection for air (or when there is no phantom present).

$${T}_{j} = \frac{{y}_{j}}{{λ}_{j , \sum }} \qquad Equation 6$$

Assuming the same photon count for air for each view angle, the quantity λ_{j,Σ} can be obtained by performing a calibration “blank scan” with all objects removed from the scanner and summing over all energy bins for each detector. A corresponding DRF defined by ϕⱼ=[ϕ_{j,0}, . . . , ϕ_{j,K-1}]∈ [symbol] ^{K} can also be defined, where ϕⱼ(p) can be given by Equation 7.

$${ϕ}_{j} ( p ) = - log \frac{{λ}_{j} ( p )}{{λ}_{j , \sum }} = - log \frac{E [ {y}_{j} \vert {p}_{j} = p ]}{{λ}_{j , \sum }} , \qquad Equation 7$$

where an assumption can be made that the function ϕⱼ(p) has been precisely measured in a calibration process for each of the scanner's detectors.

The empirically measured DRF function ϕ can account for a wide range of systematic effects that can change the observed photon count as a function of a material pathlength. The effects can include primary effects such as attenuation through the material, as well as secondary effects such as beam-hardening, photon pile-up in the detector, and Compton scatter in the detector that causes a single photon to be detected as two or more lower energy events. However, the DRF function, ϕ, does not capture effects that result from scatter in the tissue sample or other non-local effects. Using the above definitions along with the form of the Poisson distribution, an expression can be derived for the negative log likelihood, such as given by the following set of equations.

$$\begin{matrix}{f}_{j} ( p ) = {\sum }_{k = 0}^{K - 1} - log {P}_{d} ( {y}_{j , k} \vert p ) \\ = {\sum }_{k = 0}^{K - 1} {λ}_{j , k} ( p ) - {y}_{j , k} log {λ}_{j , k} ( p ) + {C}_{j} ( y ) \\ = {λ}_{j , \sum } {\sum }_{k = 0}^{K - 1} [ \frac{{λ}_{j , k} ( p )}{{λ}_{j , \sum }} - \frac{{y}_{j , k}}{{λ}_{j , \sum }} log ( {λ}_{j , k} ( p ) ) ] + {C}_{j} ( y ) \\ = {λ}_{j , \sum } {\sum }_{k = 0}^{K - 1} [ {e}^{- {ϕ}_{j , k} ( p )} + {T}_{j , k} {ϕ}_{j , k} ( p ) ] + {C}_{j}^{′} ( y )\end{matrix}$$

Dropping the constant, the loss function for the j^{th} detector can be written according to Equation 8.

$${f}_{j} ( p ) = {λ}_{j , \sum } [ {e}^{- {ϕ}_{j} ( p )} + {T}_{j} ∘ {ϕ}_{j} ( p ) ] 1 , \qquad Equation 8$$

where 1 represents a column vector of 1's, “∘” represents a Hadamard product of the vectors, and the exponential function is assumed to be applied point-wise.

Next, the calibration and computation of the photon counting detector model are described with reference to FIG. 2.

FIG. 2 illustrates example, non-limiting graphs 200 and 210 of material space sample locations used to calibrate the DRF in accordance with one or more embodiments described herein. One or more embodiments discussed with reference to FIG. 2 can be enabled by one or more components of system 100. Repetitive description of like elements and/or processes employed in respective embodiments is omitted for sake of brevity.

Non-limiting graph 200 illustrates material space sample locations used to calibrate the DRF, ϕⱼ(p)=ϕ(p; θⱼ). Non-limiting graph 200 illustrates a typical set of points used for calibration. In this case, the calibration was performed using the material bases of PE and PVC, and converting to water and bone as needed. It is evident from non-limiting graph 200 that the points are dispersed out through the two dimensional (2D) space so that an accurate polynomial fit can be computed for each detector and energy bin. Non-limiting graph 210 shows the thicknesses for slabs (or the a-space, which is space formed by pathlengths of L basis materials) used to calibrate ϕ_{θ}(p) for a center detector.

### Detector Calibration:

To model non-linear effects such as beam hardening and scatter, the DRF for the j^{th} detector and k^{th} energy bin can be represented using a low-degree polynomial denoted by ϕⱼ(p)=ϕ(p;θⱼ), where ϕ(p, θⱼ) represents a set of K polynomial functions of p∈ [symbol] ^{L} with coefficients θⱼ. More specifically, for L=2 materials, ϕ(p; θⱼ)=Σₐ₌₀⁴ Σ_{b=0}⁴ θ_{j,a,b}p₀^{a}p₁^{b}, where θⱼ is a parameter vector with K×25 components. It should be noted that coefficients θⱼ can be determined for each detector separately, and that θ_{j,a,b}∈ [symbol] ^{K} is a vector of coefficients for each energy bin. It should be further noted that for photon counting detectors with relatively narrow energy bins, ϕ_{j,k}(p) is approximately a linear function of p plus a constant offset, so the low order polynomial is appropriate, and a constant term can be included since the value of ϕ_{j,k}(0) from Equation 7 is not necessarily 0.

To estimate the coefficients of the polynomial θ_{j,k}, a set of path lengths {pₛ}ₛ₌₁^{S} can first be selected. Thereafter, for path length, pₛ, and for each detector and bin (j, k), repeated measurements of the photon counts can be taken. The repeated measurements can then be averaged to form an estimate of the expected photon count, \hat{λ}ⱼ(pₛ). From this average, an estimate of the DRF can be computed as

$${\hat{ϕ}}_{j} ( {p}_{s} ) = - log \frac{{\hat{λ}}_{j} ( {p}_{s} )}{{\hat{λ}}_{j , \sum }} .$$

Based on the estimates of the DRF, the polynomial coefficients for each detector, θⱼ, can be selected based on a least squares fit of the polynomial to the observed points/estimated points, as given by Equation 9.

$${\hat{θ}}_{j} = {arg min}_{{θ}_{j}} {\sum }_{p ∈ {p}_{s}} {‖ ϕ ( p ; {θ}_{j} ) - {\hat{ϕ}}_{j} ( p ) ‖}^{2} \qquad Equation 9$$

More generally, another approach to fitting, such as a weighted least squares or minimum cross entropy, can also be implemented.

### Fast Computation of DPM:

The following comprises a discussion of how the proximal map of Equation 3 can be efficiently computed. Since the terms are decoupled for each detector, the proximal map solution can be written in the separable form of F(p)=[F₀(p₀), . . . , F_{M-1}(p_{M-1})], where Fⱼ(pⱼ) can be given by Equation 10, and ƒⱼ(q) can be as defined in Equation 8. It is to be appreciated that the DPM can be computed at each detector in parallel.

$${F}_{j} ( {p}_{j} ) = {arg min}_{q ∈ {ℝ}^{K}} \{ {f}_{j} ( q ) + \frac{1}{2 {σ}^{2}} {‖ q - {p}_{j} ‖}^{2} \} \qquad Equation 10$$

To make the computation of the single channel DPM of Equation 10 both, fast and accurate, a majorization approach based on a quadratic surrogate function can be implemented. The exact proximal map can then be computed by iterating the surrogate update. It was observed that the DRF can be expressed in the form given by Equation 11.

$${f}_{j} ( q ) = {λ}_{j , \sum } g ( {ϕ}_{j} ( q ) ; {T}_{j} ) 1 , \qquad Equation 11$$

where the function g is assumed to use row vectors for input and output, and g can be given by Equation 12.

$$g ( z ; T ) = {e}^{- z} + T ∘ z \qquad Equation 12$$

Using Theorem 1, the optimal quadratic surrogate for the function g(z; T) over the interval [zₘᵢₙ, ∞) with base point z=z′ can be given by Equation 13.

$$g ( z ; {z}^{′} , T ) = {b}^{t} ( z - {z}^{′} ) + {( z - {z}^{′} )}^{t} \frac{c}{2} ( z - {z}^{′} ) , \qquad Equation 13$$

where

$$b = - {e}^{- z ′} + T , C = diag \{ 2 \frac{g ( {z}_{m i n} ; T ) - g ( {z}^{′} ; T ) + {g}^{′} ( {z}^{′} ; T ) ∘ ( {z}^{′} - {z}_{m i n} )}{{( {z}^{′} - {z}_{m i n} )}^{2}} \} ,$$

where the division is point-wise, and an assumption can be made that z>zₘᵢₙ in each component. Based on the assumptions, ϕ(p)>0. Thus, zₘᵢₙ can be taken to be 0 for the purposes of approximating Equation 11. However, the estimate was empirically discovered to be highly conservative and resulting in slow convergence. Since z′ is already an estimate of the minimum, zₘᵢₙ=z′−ε can be used for small ε (in experiments, ε=10⁻³ was used). Minimization with such a surrogate can be an approximate Newton step and generate faster convergence.
Theorem 1: Optimal Surrogates for Convex Functions with Concave Derivatives

Let ƒ: [xₘᵢₙ, ∞)→ [symbol] be a convex function with derivative ƒ′: [xₘᵢₙ, ∞)→ [symbol] that is concave. The for all x∈[xₘᵢₙ, ∞)→ [symbol] and x′∈(xₘᵢₙ, ∞)→ [symbol] , define Q(x; x′)=b(x−x′)+c/2(x−x′), where b and c are given by b=ƒ′(x′) and

$$c = 2 \frac{f ( {x}_{m i n} ) - f ( {x}^{′} ) + {f}^{′} ( {x}^{′} ) ( {x}^{′} - {x}_{m i n} )}{{( {x}^{′} - {x}_{m i n} )}^{2}}$$

is a surrogate function for minimization of ƒ(x) on x∈[xₘᵢₙ, ∞). Furthermore, Q provides the tightest upperbound among all quadratic surrogate functions on [xₘᵢₙ, ∞).

Using the surrogate function of Equation 13, a surrogate function for ƒⱼ(q) can be computed using a Taylor series approximation of the DRF, resulting in the following set of equations.

$$\begin{matrix}{f}_{j} ( q ; {p}^{′} ) = {λ}_{j , \sum } g ( {ϕ}_{j} ( q ) ; {ϕ}_{j} ( {p}^{′} ) , {T}_{j} ) 1 \\ = {λ}_{j , \sum } [ {b}_{j}^{t} ( {ϕ}_{j} ( q ) - {ϕ}_{j} ( {p}^{′} ) ) + \frac{1}{2} {( {ϕ}_{j} ( q ) - {ϕ}_{j} ( {p}^{′} ) )}^{t} {C}_{j} ( {ϕ}_{j} ( q ) - {ϕ}_{j} ( {p}^{′} ) ) ] \\ \approx {λ}_{j , \sum } [ {b}_{j}^{t} {A}_{j} ( q - {p}^{′} ) + \frac{1}{2} {( q - {p}^{′} )}^{t} {A}_{j}^{t} {C}_{j} {A}_{j} ( q - {p}^{′} ) ]\end{matrix} ,$$

In the above set of equations,

$${A}_{j} = ∇ {ϕ}_{j} ( {p}^{′} ) , \\ {b}_{j} = - {e}^{- {ϕ}_{j} ( {p}^{′} )} + {T}_{j} , \\ {C}_{j} = \{ 2 \frac{{e}^{- {z}_{m i n}} - {e}^{- {ϕ}_{j} ( {p}^{′} )} ∘ ( 1 + {ϕ}_{j} ( {p}^{′} ) - {z}_{m i n} )}{{( {ϕ}_{j} ( {p}^{′} ) - {z}_{m i n} )}^{2}} \} ,$$

and zₘᵢₙ=ϕ(p′; θ)−ε.

Based on the above set of equations, one partial update of the proximal map starting at p′ can be computed by introducing α=σ\sqrt{λ_{j,Σ}} and solving the following optimization.

$$\begin{matrix}{F}_{j} ( p ; {p}^{′} ) = \{ {λ}_{j , \sum } [ {b}_{j}^{t} {A}_{j} ( q - {p}^{′} ) + \frac{1}{2} {( q - {p}^{′} )}^{t} {A}_{j}^{t} {C}_{j} {A}_{j} ( q - {p}^{′} ) ] + \frac{1}{2 {σ}^{2}} {‖ q - p ‖}^{2} \} \\ = arg {min}_{q} \{ {b}_{j}^{t} {A}_{j} ( q - {p}^{′} ) + \frac{1}{2} {( q - {p}^{′} )}^{t} {A}_{j}^{t} {C}_{j} {A}_{j} ( q - {p}^{′} ) + \frac{1}{2 {α}^{2}} {‖ q - p ‖}^{2} \} \\ = {( {A}_{j}^{t} {C}_{j} {A}_{j} + \frac{1}{{α}^{2}} I )}^{- 1} [ {A}_{j}^{t} ( {C}_{j} {A}_{j} {p}^{′} - {b}_{j} ) + \frac{1}{{α}^{2}} p ]\end{matrix}$$

Algorithm 2 gives a pseudo-code algorithm for computing Fⱼ(p; p′) using N partial updates. The projection index j in the pseudocode can be suppressed for clarity. It can be observed that for a given projection, the function F(p; p′) also depends on the parameters that are specific to the detector corresponding to the projection (i.e., the transmission values, T, the total photon count, λ_{Σ}, and the calibration parameters of the detector, θ), along with parameters that are common to all the detectors (i.e., the proximal map parameter, σ, and the number of iterations, N).

### Algorithm 2: Partial Update DPM-Single Detector

Input: p, p′, T, λε, θ, σ, N

Output: F(p; p′) updated vector of path lengths
1. for i=0: N−1 do
2. zₘᵢₙ←ϕ(p′;θ)−ε
3. A←∇ϕ(p′; θ)
4. b←−e^{-ϕ(p′)}+T
5.

$$5. c ← 2 \frac{{e}^{- {z}_{m i n}} - {e}^{- ϕ ( {p}^{′} ; θ )} ∘ ( 1 + ϕ ( {p}^{′} ; θ ) - {z}_{m i n} )}{{( ϕ ( {p}^{′} ; θ ) - {z}_{m i n} )}^{2}}$$

6. C←diag{c}
7. α←σ\sqrt{Δ_{Σ}}
8.
9.

$$\begin{matrix}8. H ← {A}^{t} C A + \frac{1}{{a}^{2}} I \\ 9. q ← {A}^{t} ( {CAp}^{′} - b ) + \frac{1}{{α}^{2}} p\end{matrix}$$

10. p′←H⁻¹q
11. end for

Return: p′

Herein, Fⱼ(p; p′) can be referred to as a partial update proximal map because the algorithm can approximate the true proximal map as the number of iterations increases. However, in practice, N=1 iterations are preferable in order to reduce the computation. This can be achieved by slightly modifying the MACE algorithm of Algorithm 1 in order to provide a better initial value, pᵢₙᵢₜ, for each iteration of the MACE loop. It should be noted that for the MACE algorithm, the prior agent, H(p), may be encoded implicitly as a denoiser or other image enhancement algorithm, including a neural network denoiser. In this case, the MACE algorithm of Algorithm 1 can be used, which can reconcile multiple agents to achieve a consensus solution, \hat{p}_{MAP}. Algorithm 1 employs the Mann iteration to solve the MACE equations with a parameter ρ∈(0,1) that can be typically set to 0.8.

In Algorithm 1, p can be initialized with an MLE estimate obtained with Algorithm 3. In the initial step of Algorithm 3, a grid search can be performed for each projection (which can be done in parallel) to approximate the MLE as a starting point for the MACE algorithm. In the second for loop, ρ∈(0,1) can be a user specified parameter for which a value of 0.8 was empirically found to be sufficient. Algorithm 3 can employ the partial update proximal map to compute the maximum likelihood estimate of the path length for each detector. In this case, large values for σ and N_{MLE} can generally be used to ensure convergence to an accurate estimate of the MLE.

It is to be appreciated that due to inaccuracies in an approximation of F, Algorithm 3 can diverge for some points. In such cases, Algorithm 1 can be employed, with operator, H, set to clip p to cause the operator, H, to fall within the limits of the material space in non-limiting graph 200. After computation of the MACE or MLE estimates, the final image can be reconstructed using 3D FDK reconstruction on the components of p.

### Algorithm 3: MLE Algorithm

Input: T, λ_{Σ}, θ, N_{MLE}, σ, N_{sub}=1

Output: p the material path lengths
1. for j=0: M−1 do
2. pⱼ←GridSearch(argminₚ{Σₖ e^{-ϕ}^{_{j,k}}^{(p;θ}^{ⱼ}⁾+ϕ_{j,k}(p; θⱼ)T_{j,k}})
3. end for
4. for i=0: N_{MLE}−1 do
5. p←F(p; p, T, λ_{Σ}, θ, σ, N_{sub})

end for

Return: p

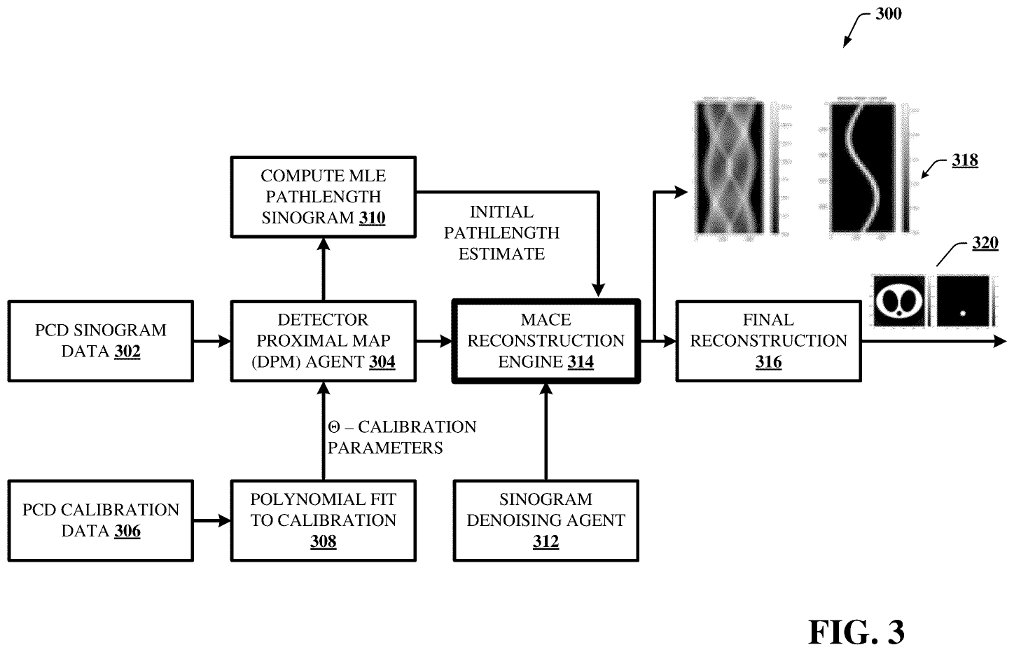

FIG. 3 illustrates a flow diagram of an example, non-limiting method 300 that can perform modular material decomposition from energy resolving photon counting data in accordance with one or more embodiments described herein. One or more embodiments discussed with reference to FIG. 3 can be enabled by one or more components of system 100. Repetitive description of like elements and/or processes employed in respective embodiments is omitted for sake of brevity.

Various embodiments herein disclose a modular algorithm for computationally efficient material decomposition using CT measurements obtained with a photon counting detector. The modular algorithm can use a MACE framework to balance two complementary update operators, or agents, namely, a data-fitting agent (or forward model) and a prior agent. The data-fitting agent can use an accurate surrogate function approximation to the detector response to produce an approximate proximal map that can access a candidate reconstruction as input and produce a reconstruction that has a better fit to data. The prior agent can also access a candidate reconstruction and return a reconstruction with better image properties, typically in the form of reduced noise. The two agents (i.e., the data-fitting agent or forward model and prior agent) can be applied sequentially, along with a Lagrangian communication term to enforce a common solution, until convergence. The MACE algorithm can allow for each of the two agents to be designed and implemented separately and combined into a computationally efficient and modular reconstruction algorithm. With the MACE framework, the proposed algorithm can use material-specific constraints and advanced, pre-trained prior agents, either in the projection domain or in the image domain. The embodiments discussed herein are focused on processing entirely in the projection domain followed by a single reconstruction step to promote computational efficiency, but the modularity of the method disclosed by embodiments of the present disclosure implies that the use of priors in the image domain can be a straightforward variation.

The mechanism behind CT involves combining X-ray technology with computer processing by projecting X-rays through an object (e.g., a 3D object, anatomy, etc.) and collecting/detecting photons at the other end of the object via a detector, followed by reconstructing a 3D volume from different views of the object from different angles. X-rays being a form of electromagnetic radiation, the different views can be generated by collecting and counting the photons received by the detector. Different photons have different energies. In non-PCD based CT, an energy integrating detector (EID) can measure an amount of energy associated with photons for a period of time and generate a single number as an output based on the measurement. On the contrary, a PCD does not measure the total amount of energy from the different energies or wavelengths of different photons but measures the number of photons. A PCD can measure the number of photons at multiple different frequencies or wavelengths of the photons. As a result, a PCD-based CT can generate a finer grained look at an object (e.g., a 3D object, anatomy, etc.) being examined.

In non-limiting method 300, MACE reconstruction engine 314 represents the MACE algorithm (e.g., reconstruction component 108). MACE reconstruction engine 314 can use data from a PCD-based CT and reconstruct a representation of a 3D object in multiple materials. For example, MACE reconstruction engine 314 can ingest photon counting data with the goal of reconstructing 3D representation data of an object in terms of two basis materials, wherein the basis materials can often be water and bone, or water and iodine, PE and PVC, etc. The basis materials can be selected based on the two different ways in which a photon can interact with any material so that reconstructed 3D representation data can be distinguished from one another. For example, in X-ray technology, a photon can interact with an object in two primary ways, namely, the photoelectric effect and the Compton effect. As a result, a material can be identified as a ratio between the two effects based on which other properties of the original object can be estimated. The two basis materials can provide a good representation of a manner in which an object can interact with X-rays.

In a PCD-based CT system, the PCD can comprise photon bins, and each photon bin can correspond to a different frequency range. Thus, a CT system with eight photon bins in the PCD (e.g., as employed in connection with various embodiments herein) can have eight different ranges of frequencies for the photons. As a result, an eight dimensional (8D) measurement can be obtained at each point because the CT system can generate respective numeric values for each one of the eight different bins, wherein each numeric value can represent the projection of the object onto a photon bin of the PCD. Thereafter, different views can be generated that can be used by MACE reconstruction engine 314 to construct a 3D volume and each point in the 3D volume can have two numeric values, one for each basis material.

In non-limiting method 300, the set of images 320 illustrates 3D volumes reconstructed in terms of two basis materials. The left image in the set of images 320 illustrates a cross section of the lungs with a spine, two lung cavities and the surrounding chest area. The set of images 318 illustrates material sinograms. Sinograms are data that can be generated from the measured data (i.e., photon counting data) from a PCD, and measured data from each energy bin of the PCD can be used to generate a respective sinogram. Thus, eight different sinograms corresponding to different energy/frequency bins can be generated from measured data from a CT system with eight photon bins. In non-limiting method 300, the sinograms generated from PCD data are represented by PCD sinogram data 302. In various embodiments herein, MACE reconstruction engine 314 can use such multidimensional (e.g., 8D) sinograms and generate the set of images 318 by combining the multidimensional sinograms with additional information about the data collected by the CT system and information about what a good sinogram should look like. That is, MACE reconstruction engine 314 can ingest PCD sinogram data 302 comprising multidimensional (e.g., 8D) sinograms corresponding to photon counts and convert the sinograms into the set of images 318 comprising two dimensional sinograms corresponding to material pathlengths.

MACE reconstruction engine 314 can ingest two sources of algorithmically defined information. For example, in various embodiments herein, generation component 112 can generate two agents that can be employed along with MACE reconstruction engine 314 to generate the set of images 318. Of the two agents, DPM agent 304 can ingest a low quality version of the material sinograms illustrated by the set of images 318 to improve upon the low quality version of the material sinograms. DPM agent 304 can perform such an improvement by converting the low quality version of the set of images 318 into a better match to PCD sinogram data 302. DPM agent 304 can also perform the improvement by accessing material sinograms, comparing the material sinograms to PCD sinogram data 302 and moving the material sinograms closer to fitting the data better, without deviating too far away from the input to DPM agent 304 which can include a current estimate of the sinograms. Of the two agents generated by generation component 112, sinogram denoising agent, 312 can access the material sinograms and improve upon an appearance of the material sinograms. For example, material sinograms can comprise noise that can cause undesirable artefacts in the material sinograms, and sinogram denoising agent 312 can remove some of the undesirable artefacts from the material sinograms while retaining useful information. In some embodiments, sinogram denoising agent 312 can be another type of agent directed to a purpose other than denoising. That is, in some embodiments, generation component 112 can generate a different agent instead of sinogram denoising agent 312 for other types of image processing.

As described above, PCD sinogram data 302 can be representative of eight bins of photons for different frequency ranges (e.g., low frequency photons, medium frequency photons, high frequency photons, etc.) and the PCD can collect such data and measure the collect data at individual frequencies. The eight sinograms generated from data collected by the PCD and corresponding to individual photon bins of the PCD can appear similar to the left hand side material sinogram in the set of images 318, and the respective sinograms can have different hot spots, varying levels of noise, etc. from one another.

For executing the transformation from frequencies to material pathlengths, information about what a CT scanner will record for different combination of the two basis materials can be determined. For example, a technician can load a CT scanner with defined amounts of the two basis materials (e.g., PE and PVC). Thereafter, a range/length of each of the two materials can be recorded, followed by stacking the two basis materials and taking a scan, wherein the scan can record frequencies for the eight energy bins associated with the combination of materials loaded into the CT scanner. Seventy-eight combinations of different respective lengths of each of the two basis materials can be considered by independently varying the individual lengths of each basis material to generate seventy-eight different calibration points as PCD calibration data 306. Thus, each calibration point can correspond to two different lengths, one corresponding to each basis material, and an input to the CT scanner comprising a combination of the two basis materials taken at their respective lengths can generate eight numeric values for each detector in the CT scanner, wherein the eight numeric values represent eight different measurements of the photons for the corresponding energy bins. Based on this data, various embodiments herein can aim to evaluate a correct frequency response for several different material combinations, including combinations not calibrated for. For doing so, polynomial fit to calibration 308 can be used, wherein polynomial fit to calibration 308 can be a function that can approximate PCD calibration data 306 and extrapolate or extend PCT calibration data 306 to generate values even for length combinations of the two basis materials for which scans have not been generated.

DPM agent 304 can use polynomial fit to calibration 308, which can be encapsulated in the calibration parameters. In non-limiting method 300, theta (θ) can represent the set of calibration parameters that can be obtained from the calibration data and used in DPM agent 304. θ can be combined with PCD sinogram data 302 to determine, using DPM agent 304, a likelihood of a certain combination of basis materials relative to measured values of the frequency data/photon data, at each given measurement location. For a given set of measurements and calibration data, the most likely set of pathlengths that can correspond to each of eight photon measurements can be determined based on a Poisson probability distribution. The Poisson probability distribution can represent a physical model of what the measurements can look like based on the physics of photons. For a certain measured data (i.e., the photon counting data from a PCD), material pathlengths for basis materials, which material pathlengths can be most likely to have produced a measurement, can be determined for each point by compute MLE pathlength sinogram 310. Some existing techniques can use MLE directly to reconstruct CT images; however, as evident from experimental results presented with reference to subsequent figures, embodiments of the present disclosure generate better outcomes as compared to a direct implementation of MLE. Based on the set of images 318, final reconstruction 316 can be performed by an algorithm to generate the set of images 320.

In some embodiments, DPM agent 304, polynomial fit to calibration 308, compute MLE pathlength sinogram 310, sinogram denoising agent 312, MACE reconstruction engine 314, and final reconstruction 316 can be or can be performed by individual neural networks or machine learning operators or models. In such embodiments, the blocks DPM agent 304, polynomial fit to calibration 308, compute MLE pathlength sinogram 310, sinogram denoising agent 312, MACE reconstruction engine 314, and final reconstruction 316 can represent individual stages of a multi-stage machine learning model that can reconstruct material decomposed CT images from photon counting data. In general, the techniques described herein can be applied with various different neural networks trained in many different ways. For example, in an embodiment, sinogram denoising agent 312 can be a neural network that can self-train on CT images to perform denoising, without needing training. In another embodiment, data collected over time can be used to uncover statistics that can be used to train one or more neural networks employed for reconstructing material decomposed CT images. In yet another embodiment, DPM agent 304 and sinogram denoising agent 312 can be implemented as individual neural networks while running the same algorithm in the same way. For example, in Equation 4, H can be replaced by a neural network that can perform denoising or other image improvement. Therefore, it is to be appreciated that Equations 3 and 4 presented supra can represent only one way of implementing DPM agent 304 and sinogram denoising agent 312. In general, embodiments of the present disclosure can enable a modular framework for reconstructing material decomposed CT images.

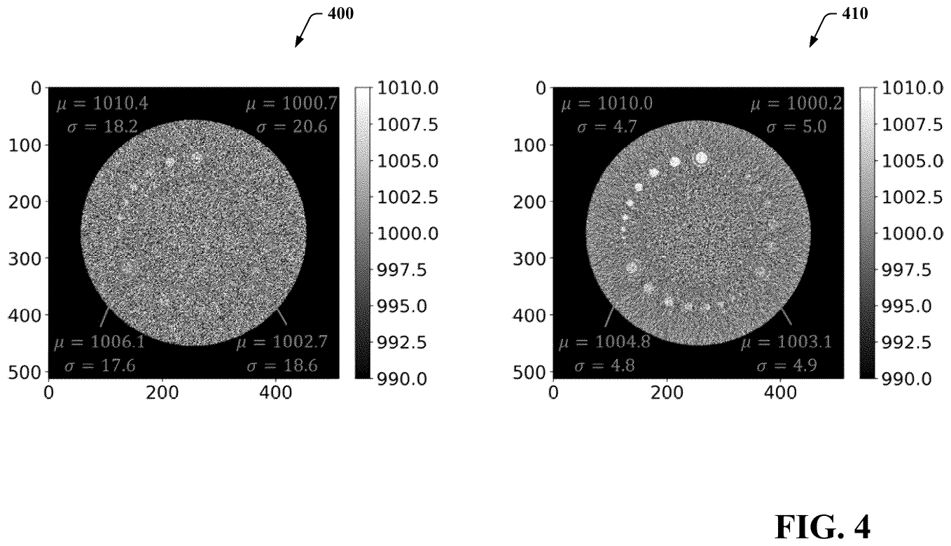

FIG. 4 illustrates example, non-limiting reconstructions 400 and 410 formed as a linear combination of material decomposed reconstructions weighted to simulate a 70 keV monoenergetic scan in accordance with one or more embodiments described herein. Repetitive description of like elements and/or processes employed in respective embodiments is omitted for sake of brevity.

FIG. 4 illustrates reconstructions of a simulated water background (density 1.0) with inserts of densities 1.01, 1.005, and 1.003. The results are displayed in Hounsfield units (HU) plus 1000, so that water is 1000 and air is 0. Non-limiting reconstruction 400 illustrates a reconstruction from an MLE pathlength sinogram. Non-limiting resonctruction 410 illustrates a reconstruction from a MACE-denoised sinogram showing an increased CNR (0.5 vs 2.0) and reduced noise. Non-limiting reconstructions 400 and 410 are formed as a linear combination of material decomposed reconstructions, weighted to simulate a 70 keV monoenergetic scan.

The proposed algorithm (i.e., MACE algorithm) was evaluated with simulated and measured photon counting data. The simulated data (spectrally binned, simulated photon counting data) was collected using General Electric (GE) HealthCare's CatSim software. The measured data was collected and processed separately. Both datasets (i.e., the simulated data and the measured data) were collected for a detector with 2500 columns, 2 rows and 8 energy bins. The simulated detector can model effects due to Compton scatter in detector channels. However, the effects of object scatter, charge sharing, and crosstalk among detectors were not considered in the simulation.

In the experiments performed in connection with the various embodiments disclosed herein, two basis materials were considered owing to two significant interactions of X-rays used in clinical CT with matter (photoelectric and Compton effects). PE and PVC were selected as basis materials for the ease of data collection for calibration. The proposed algorithm produced fractional volume maps for the two basis materials. Using the estimated maps, a virtual mono-energy reconstruction was computed at 70 keV. Using the volume fractions estimated for PVC and PE, a virtual mono-energy reconstruction at energy E keV was computed using \hat{x}_{E}^{(joint)}=μ₁(E)\hat{x}_{*,1}+μ₂(E)\hat{x}_{*,2}, where μₗ(E) is the linear attenuation coefficient for the l^{th} basis material at energy E keV. When needed, the estimated maps were converted to report fraction volume maps for water and bone using a linear transformation.

### Calibration:

Calibrating ϕ_{θ}(p) for each detector channel can utilize pairs (p, ϕ(p)), where each row in p∈ [symbol] ^{M}^{_{C}}^{×L} can be a point in a-space (i.e., space formed by pathlengths of L basis materials) used in calibration, M_{C} can represent the number of data points for calibration, and ϕ(p) can represent the measured negative log attenuation for a corresponding p. Pairs (p, ϕ(p)) can be generated by scanning slabs of PE and PVC of known thicknesses for a single view (X-ray source at 0°). To calibrate the DRF ϕ(p; 0), slabs of PE and PVC of thicknesses equal to the selected set of path lengths, {pₛ}ₛ₌₁^{S}, shown in non-limiting graph 200 of FIG. 2, were scanned sequentially for a single view (X-ray source at 0°) multiple times. The slabs were kept perpendicular to the iso-ray (line connecting the source and iso) and covered the entire detector. Non-limiting graph 210 of FIG. 2 shows the thicknesses for slabs (or the a-space) used to calibrate ϕ_{θ}(p) for a center detector. It should be noted that path lengths at off-center detector channels were adjusted to account for the scanner geometry. To obtain a noise-free ϕ(p), noise can be turned off in simulation. For measured data, multiple scans can be averaged. Using the generated pairs (p, ϕ(p)), a least-squares solution can be obtained for each detector.

### Datasets:

For experiments with simulated data, a phantom with inserts of densities close to water was used to evaluate performance on low-contrast targets. The phantom was scanned 12 times at a dosage 120 Kilovoltage peak (kVp)/400 Milliampere (mA) at a rotation speed of 1 second(s) per rotation for 1000 views and averaged the reconstruction results. The scan was reconstructed for the image size of 512×512 and Display-Field-of-View (DFOV) of 25.6 cm. For experiments with measured data, data obtained from scans of GE's small gammex and lungman phantoms was used. The small gammex contained iodine inserts with various densities. The DFOV was 40 cm which was then cropped to a size of 512×512 to show only the relevant region. The DFOV for the lungman phantom was 35 cm. The lungman phantom mimics a human chest scan. The scans were reconstructed for an image size of 1024×1024. For the small gammex phantom, a full field of view reconstruction of 40 cm was performed. This type of reconstruction has a significant amount of blank space surrounding the phantom. Thus, the reconstruction was followed by cropping the image to a 512×512 image showing only the gammex phantom, which resulted in the DVOF being close to 21 cm for the final image shown for the small gammex. A similar technique was adopted for the lungman phantom with the 35 cm and 1024×1024 size.

In various embodiments, an anisotropic denoiser can be employed for the denoising agent to enhance a quality of a MACE reconstruction. The anisotropic denoising agent can ingest an input of a sinogram using the base material coordinate system. The anisotropic denoiser can then rotate a 2D or an L dimensional (L-D) material space, wherein L can be a positive integer, into coordinates associated with high and low measurement uncertainty. The coordinate rotation can be determined empirically or adaptively by measuring or calculating analytically the noise covariance in the material decomposition space. Once the sinogram is rotated to these orthogonalized coordinates, the two components can be denoised using different levels of denoising. Typically, the high variance component can be more strongly denoised with, for example, a larger filter point spread function (psf), and the lower variance component can be more weakly denoised with, for example, a smaller filter point-spread function. Such denoisers can be linear or nonlinear. Once the two components are denoised, the two components can be rotated back into the original base material coordinate system and returned by the anisotropic denoising agent. In experiments, employing the anisotropic denoiser can substantially improve the reconstructions.

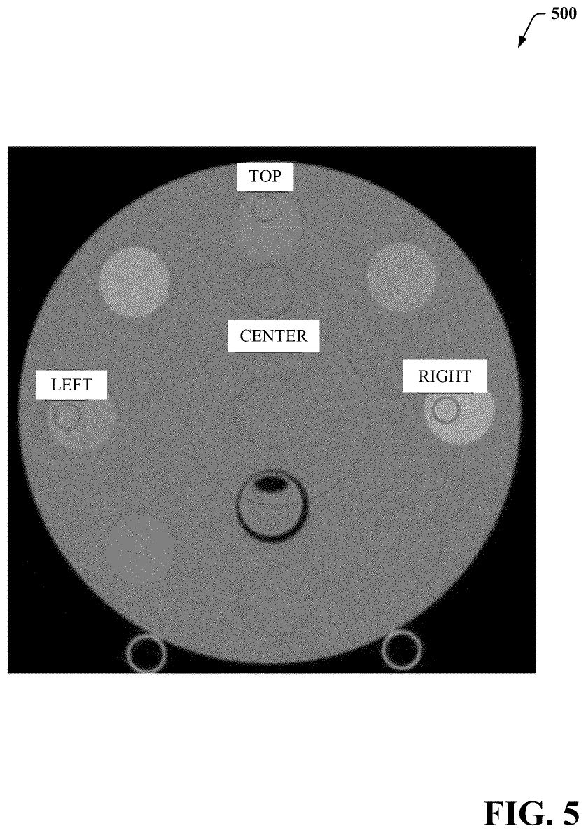

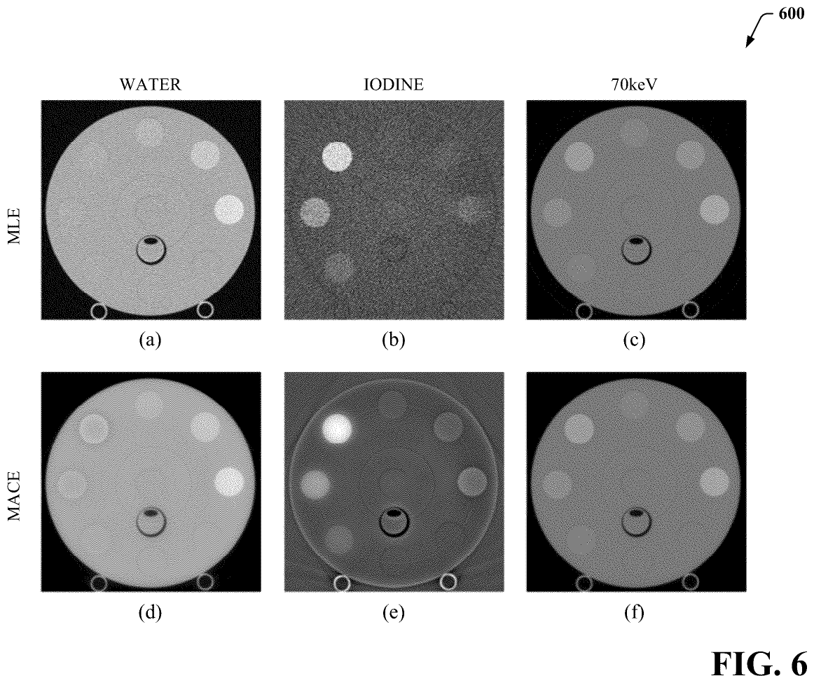

FIG. 5 illustrates an example, non-limiting reconstruction 500 of a small gammex in accordance with one or more embodiments described herein. FIG. 6 illustrates an example, non-limiting set 600 of MLE and MACE reconstructions for measured data with a small gammex phantom in accordance with one or more embodiments described herein. Repetitive description of like elements and/or processes employed in respective embodiments is omitted for sake of brevity.

As described supra, FIG. 4 illustrates the baseline MLE reconstruction on the left and the reconstruction using the MACE algorithm on the right. The reconstructions are illustrated in HU, modified so that air is 0 and water is 1000, with a center of 1000 HU and a window of 20 HU. The result based on the techniques proposed by embodiments of the present disclosure was generated by utilizing a simple linear filter as a prior, but the MACE algorithm was still able to distinguish inserts with less than 0.3% deviation from water density, even at very small feature lengths. The linear filter was used in the channel direction only on both the water and bone sinogram.

Non-limiting reconstruction 500 of FIG. 5 labels circles used for quantitative evaluation. FIG. 6 illustrates a reconstruction for the small gammex with the proposed algorithm (bottom row) as well as a comparison to the calculated MLE (top row). Non-limiting reconstruction 500 illustrates a reconstruction of a small gammex. The circles in non-limiting reconstruction 500 correspond to the names and locations of the mean and standard deviation calculated in Table 1 on the reconstructions in FIG. 6. As such, Table 1 lists the means and standard deviations for the selected circles. As expected from FIG. 6, the method for reconstruction of material decomposed CT images generates a similar mean but a significant reduction in standard deviation compared to the MLE quantitatively.

More specifically, non-limiting set 600 of FIG. 6 illustrates MLE and MACE reconstructions for measured data with a small gammex phantom. The MACE algorithm can delineate inserts with various densities. FIG. 6 illustrates a water display window of [0, 1450] HU, an Iodine display window of [−50, 110] HU, and a 70 keV display window [0, 2000] HU. The MLE estimates were obtained with N_{MLE}=100 to ensure convergence. In various embodiments, the proposed method can also employ an MLE estimate to initialize p. However, in this case, N_{MLE}=15 was found to be sufficient, saving on computation time. Thus, the number of iterations needed can be reduced to calculate an approximate value for the MLE while still achieving significant noise reduction. It should be noted that for the results with measured data, for the prior agent, the Gaussian psf was first applied. Thereafter, the values were also clipped to the limits of the calibration space shown in non-limiting graph 200. Non-limiting set 600 illustrates a visual comparison in noise between the MLE and the MACE results. As evident, FIG. 6 reconstructions have thin, bright and dark concentric rings, which represent artifacts due to “bad” detectors. The bad detector measurements can be removed in practice. The techniques disclosed in the various embodiments herein provide a flexibility to scale the λ_{Σ} parameter in a band around the “bad” detectors to remove such artifacts. The fine tuning of λ_{Σ} can be performed separately and was not performed for the results displayed in FIG. 6.

Table 1: Mean and standard deviation for reconstructions in FIG. 6 and circles named in FIG. 5

| Material | Circle | MLE | MACE |
|---|---|---|---|
| Water | Center | 966.21 | 960.64 |
| | Left | 994.93 | 986.41 |
| | Right | 1254.70 | 1244.07 |
| | Top | 1035.07 | 1030.82 |
| Iodine | Center | 1.21 | 2.20 |
| | Left | 48.31 | 49.16 |
| | Right | 18.74 | 19.19 |
| | Top | 3.92 | 4.59 |
| 70 keV | Center | 968.84 | 965.39 |
| | Left | 1099.39 | 1092.69 |
| | Right | 1295.22 | 1285.57 |
| | Top | 1043.54 | 1040.75 |

(a) Mean

| Material | Circle | MLE | MACE |
|---|---|---|---|
| Water | Center | 176.36 | 15.28 |
| | Left | 171.56 | 15.86 |
| | Right | 194.03 | 15.39 |
| | Top | 159.72 | 13.83 |
| Iodine | Center | 59.05 | 3.54 |
| | Left | 57.57 | 3.77 |
| | Right | 64.01 | 3.87 |
| | Top | 52.22 | 3.11 |
| 70 keV | Center | 79.28 | 18.18 |
| | Left | 74.41 | 18.44 |
| | Right | 83.73 | 17.67 |
| | Top | 72.93 | 18.00 |

(b) Standard Deviation

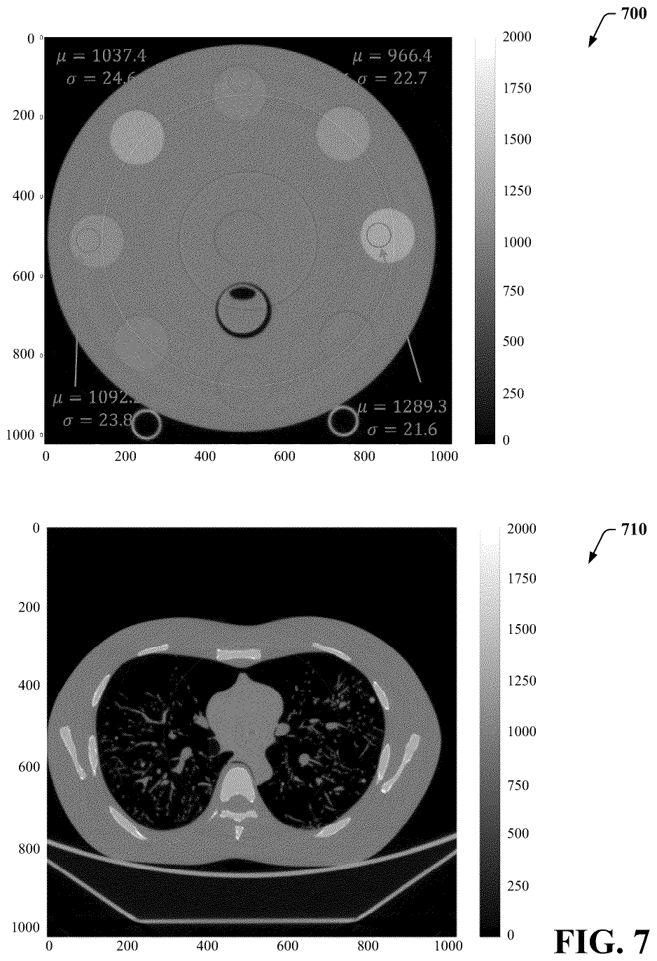

FIG. 7 illustrates example, non-limiting MACE reconstructions 700 and 710 for measured data with a small gammex phantom and a lungman phantom in accordance with one or more embodiments described herein. Repetitive description of like elements and/or processes employed in respective embodiments is omitted for sake of brevity.

Various embodiments herein can provide a material decomposition algorithm that can perform a joint optimization in projection and image domain leveraging information in both domains. The joint optimization can be computationally expensive if solved directly. Therefore, various embodiments herein can employ a surrogate function for a detector log likelihood and a MACE algorithm to make the optimization computationally efficient and modular. With the MACE framework, the MACE algorithm can use advanced, pre-trained prior agents and material-specific constraints. A variation of the MACE algorithm can perform optimizations entirely in the projection domain, further reducing the computational complexity.

With continued reference to FIG. 4, non-limiting MACE reconstruction 700 of FIG. 7 illustrates a MACE reconstruction for measured data with the small gammex phantom using the MACE algorithm. As noted before, the MACE algorithm can delineate inserts with various densities. That is, the method disclosed herein can clearly resolve inserts with small differences in densities relative to the background. The measured data included measurements from bad pixels that lead to thin, bright and dark concentric rings. The bad pixel measurements can be removed in practice. Non-limiting MACE reconstruction 710 illustrates a MACE reconstruction for measured data with the lungman phantom. As evident from non-limiting MACE reconstruction 710, the MACE algorithm can denoise reconstructions while retaining detail and uniform texture. That is, the MACE algorithm can achieve a low noise reconstruction while maintaining sharp image detail.

As stated elsewhere herein, the framework provided by various embodiments of the present disclosure can provide an efficient reconstruction method to produce CT reconstructions with material decomposition from PCD data. The modularity of the system (e.g., system 100) can be valuable in that it can allow for an easy modification to better model a wide variety of detectors and system geometry, as well as to utilize advanced prior models, including algorithmic priors such as denoisers or neural networks, either in the sinogram domain or the image domain. In this regard, results generated from experiments on simulated data indicate an excellent 4.5 times boost in CNR on small low contrast phantom inserts, corresponding to a dose saving of over 20 times. In prototype form, the MACE algorithm can need under 2.5 seconds(s) of computation per detector row (native slice), which can indicate that the MACE algorithm can be suitable for a practical implementation in clinical PCCT systems that are typically deployed with extensive parallel processing resources. Additionally, the techniques disclosed herein are proven to work and perform well with real Pluto data. In this regard, the techniques disclosed herein also differ from the current Pluto MD-based imaging chain and can overcome potential boundary artifact issues.

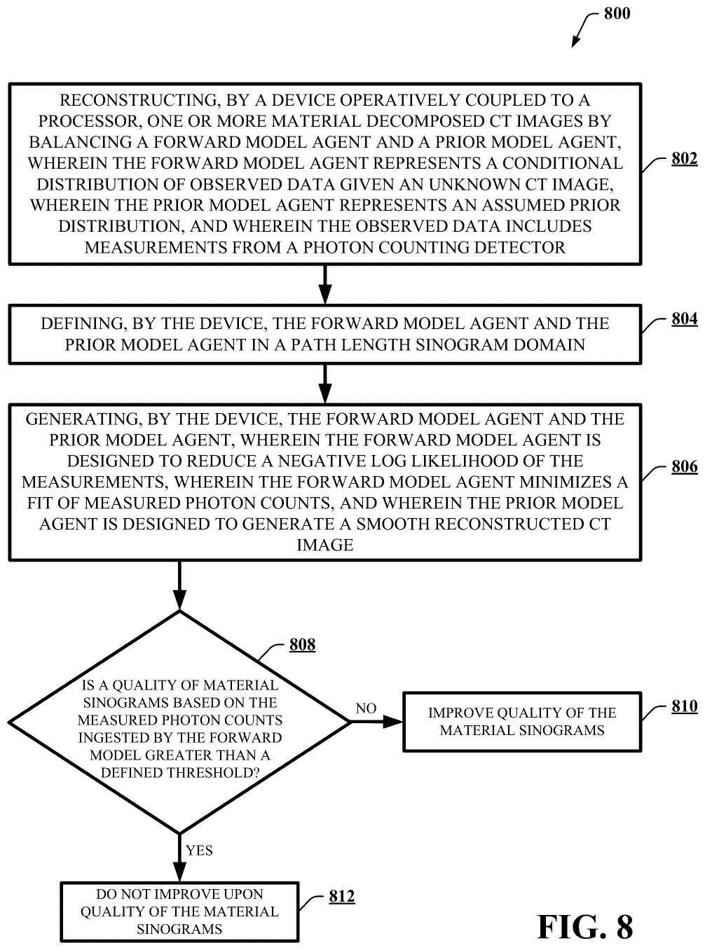

FIG. 8 illustrates a flow diagram of an example, non-limiting method 800 that can perform modular material decomposition from energy resolving photon counting data in accordance with one or more embodiments described herein. One or more embodiments discussed with reference to FIG. 2 can be enabled by one or more components of system 100. Repetitive description of like elements and/or processes employed in respective embodiments is omitted for sake of brevity.

At 802, the non-limiting method 800 can comprise reconstructing (e.g., by reconstruction component 108), by a device operatively coupled to a processor, one or more material decomposed CT images by balancing a forward model agent and a prior model agent, wherein the forward model agent can represent a conditional distribution of observed data given an unknown CT image, wherein the prior model agent can represent an assumed prior distribution, and wherein the observed data can include measurements from a photon counting detector.

At 804, the non-limiting method 800 can comprise defining (e.g., by definition component 110), by the device, the forward model agent and the prior model agent in a path length sinogram domain.

At 806, the non-limiting method 800 can comprise generating (e.g., by generation component 112), by the device, the forward model agent and the prior model agent, wherein the forward model agent can be designed to reduce a negative log likelihood of the measurements, wherein the forward model agent can minimize a fit of measured photon counts, and wherein the prior model agent can be designed to generate a smooth reconstructed CT image.

At 808, the non-limiting method 800 can comprise determining whether a quality of material sinograms based on the measured photon count ingested by the forward model is greater than a defined threshold. If no, at 810, the non-limiting method 800 can comprise improving upon quality of the material sinograms by using the forward model. If yes, at 812, the non-limiting method 800 can comprise not improving upon quality of the material sinograms by using the forward model.

For simplicity of explanation, the computer-implemented and non-computer-implemented methodologies provided herein are depicted and/or described as a series of acts. It is to be understood that the subject innovation is not limited by the acts illustrated and/or by the order of acts, for example acts can occur in one or more orders and/or concurrently, and with other acts not presented and described herein. Furthermore, not all illustrated acts can be utilized to implement the computer-implemented and non-computer-implemented methodologies in accordance with the described subject matter. Additionally, the computer-implemented methodologies described hereinafter and throughout this specification are capable of being stored on an article of manufacture to enable transporting and transferring the computer-implemented methodologies to computers. The term article of manufacture, as used herein, is intended to encompass a computer program accessible from any computer-readable device or storage media.

The systems and/or devices have been (and/or will be further) described herein with respect to interaction between one or more components. Such systems and/or components can include those components or sub-components specified therein, one or more of the specified components and/or sub-components, and/or additional components. Sub-components can be implemented as components communicatively coupled to other components rather than included within parent components. One or more components and/or sub-components can be combined into a single component providing aggregate functionality. The components can interact with one or more other components not specifically described herein for the sake of brevity, but known by those of skill in the art.

One or more embodiments described herein can employ hardware and/or software to solve problems that are highly technical, that are not abstract, and that cannot be performed as a set of mental acts by a human. For example, a human, or even thousands of humans, cannot efficiently, accurately and/or effectively reconstruct material decomposed CT images as the one or more embodiments described herein can enable this process. And, neither can the human mind nor a human with pen and paper process photon counting data from a PCD-based CT scanner to reconstruct the material decomposed CT images, as conducted by one or more embodiments described herein.

Realizing the full potential of PCCT can involve advanced denoising algorithms to manage the noise vs spatial resolution trade-off so that ultra-high resolution images may be produced at acceptable doses. Doing so can enable detection of, for example, small, low-contrast structures that are not visible on contemporary CT images. As stated elsewhere herein, embodiments of the present disclosure demonstrate a CNR boost of 4.5 times relative to the MLE baseline in a phantom study using a simulator that can realistically model a Silicon-detector-based photon counting clinical prototype. If the 20 times dose reduction can be realized in actual CT clinical systems, high resolution and ultra-low-dose screening applications of PCCT can be facilitated. In addition, the embodiments disclosed herein can employ a forward model that can be used to probabilistically map photon interactions in the spectral tail (e.g., from Compton scatter, K-escape fluorescence and charge sharing) into primary bins. Together with the modular structure of the MACE framework, this can offer great flexibility for detector modeling and model calibration

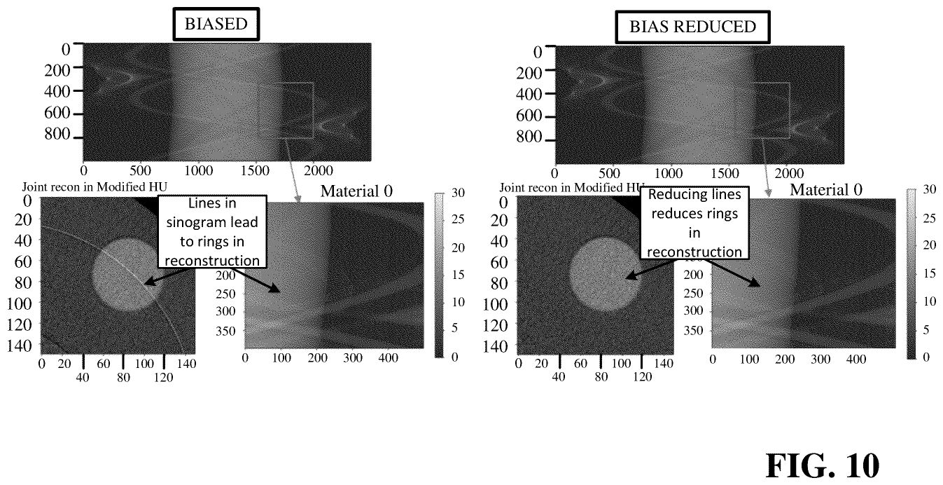

In some examples, ideal detectors are presumed. In practice, detectors can sometimes have imperfectly calibrated detector cells. These imperfect calibrations are seen as vertical lines in a sinogram which can lead to rings in the reconstruction as shown in FIG. 9, which illustrates an example reconstruction with bad detectors. The red arrows show where two rings are present due to these poor detectors. One way to reduce artifacts present from rings is to calculate an estimation of the bias from the sinogram and remove it in one of the MACE agents. FIG. 10 illustrates a relationship between imperfectly calibrated detector cells in a sinogram and rings in the reconstruction. It also shows that reducing bias can correct these detectors and reduce ringing artifacts. As seen in FIG. 10, which is an example of poor detector creating rings in reconstruction. The left shows the biased sinogram with a bad detector creating the ring. The right shows when bias is reduced the detector is corrected and the ring is removed.

If we let θ be the parameters of the system, then bias is defined as the difference between the true value of the parameters and the expected value of the estimator, or Bias=θ−E(θ\hat{ }) where θ\hat{ } is an estimate of θ. Given a view, we expect nearby detectors to yield similar photon counts. When averaging across views, if we find detectors drastically different from their neighbors, this shows bias in our estimate. This assumption will motivate our calculation of bias described in the rest of the section.

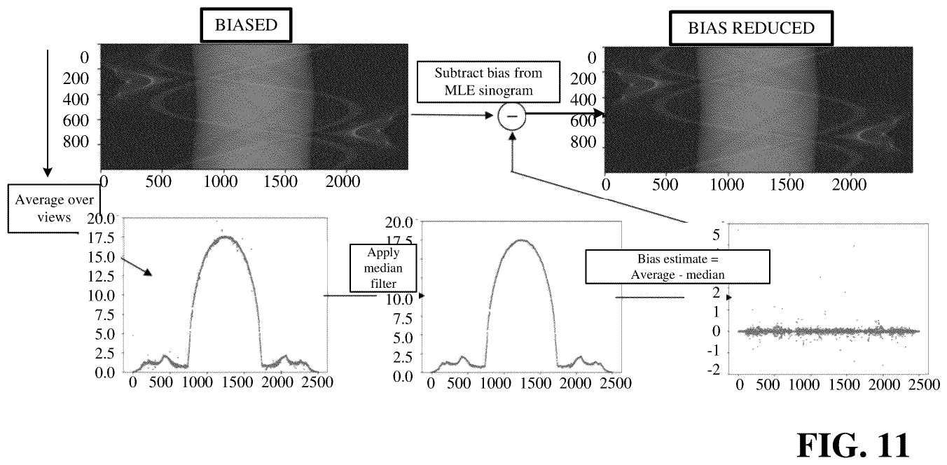

FIG. 11 illustrates an example of reducing the bias of the sinogram. In this case, we can calculate an estimate of the bias by averaging the MLE sinogram across views and applying a filter to correct the imperfectly calibrated detector cells. In our case, we use a median filter across the detector columns, but in practice, more sophisticated means of anomaly detection can be used. After applying the filter, we can subtract the filtered version from the averaged version to give an estimate of the bias. FIG. 11 shows this with an example. More precisely, let pΣR\hat{ }(M×C×R×L) where M is number of projections, C is number of detector columns, R is number of detector rows, and Lis number of materials. Then an estimate of the bias is

E[p]−MedFilt[E[p]].

We can implement this bias reduction step by adding it to either agent, but we implement it in the forward agent. This allows the denoiser to operate on a bias reduced sinogram and reduces the propagation of the imperfect calibration. The consensus equilibrium solves the problem

F(p)=H(p)

where F and H are the forward model and prior model. With the bias reduction step, we want to solve the problem

B⁻¹F(Bp)=H(p)

where B is an operator that takes an unbiased sinogram and adds the bias back. This means B⁻¹ reduces the bias of a biased sinogram.

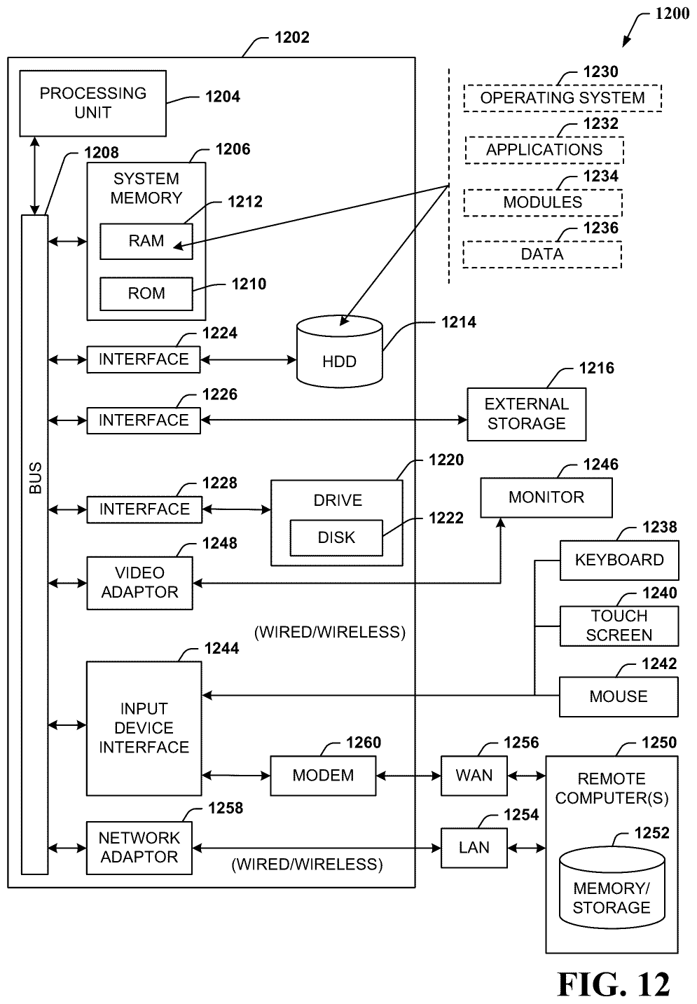

In order to provide additional context for various embodiments described herein, FIG. 12 and the following discussion are intended to provide a brief, general description of a suitable computing environment 1200 in which the various embodiments described herein can be implemented. While the embodiments have been described above in the general context of computer-executable instructions that can run on one or more computers, those skilled in the art will recognize that the embodiments can be also implemented in combination with other program modules or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multi-processor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 12, the example environment 1200 for implementing various embodiments of the aspects described herein includes a computer 1202, the computer 1202 including a processing unit 1204, a system memory 1206 and a system bus 1208. The system bus 1208 couples system components including, but not limited to, the system memory 1206 to the processing unit 1204. The processing unit 1204 can be any of various commercially available processors. Dual microprocessors and other multi-processor architectures can also be employed as the processing unit 1204.

The system bus 1208 can be any of several types of bus structure that can further interconnect to a memory bus (with or without a memory controller), a peripheral bus, and a local bus using any of a variety of commercially available bus architectures. The system memory 1206 includes ROM 1210 and RAM 1212. A basic input/output system (BIOS) can be stored in a non-volatile memory such as ROM, erasable programmable read only memory (EPROM), EEPROM, which BIOS contains the basic routines that help to transfer information between elements within the computer 1202, such as during startup. The RAM 1212 can also include a high-speed RAM such as static RAM for caching data.

The computer 1202 further includes an internal hard disk drive (HDD) 1214 (e.g., EIDE, SATA), one or more external storage devices 1216 (e.g., a magnetic floppy disk drive (FDD) 1216, a memory stick or flash drive reader, a memory card reader, etc.) and a drive 1220, e.g., such as a solid state drive, an optical disk drive, which can read or write from a disk 1222, such as a CD-ROM disc, a DVD, a BD, etc. Alternatively, where a solid state drive is involved, disk 1222 would not be included, unless separate. While the internal HDD 1214 is illustrated as located within the computer 1202, the internal HDD 1214 can also be configured for external use in a suitable chassis (not shown). Additionally, while not shown in environment 1200, a solid state drive (SSD) could be used in addition to, or in place of, an HDD 1214. The HDD 1214, external storage device(s) 1216 and drive 1220 can be connected to the system bus 1208 by an HDD interface 1224, an external storage interface 1226 and a drive interface 1228, respectively. The interface 1224 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1202, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1212, including an operating system 1230, one or more application programs 1232, other program modules 1234 and program data 1236. All or portions of the operating system, applications, modules, or data can also be cached in the RAM 1212. The systems and methods described herein can be implemented utilizing various commercially available operating systems or combinations of operating systems.

Computer 1202 can optionally comprise emulation technologies. For example, a hypervisor (not shown) or other intermediary can emulate a hardware environment for operating system 1230, and the emulated hardware can optionally be different from the hardware illustrated in FIG. 12. In such an embodiment, operating system 1230 can comprise one virtual machine (VM) of multiple VMs hosted at computer 1202. Furthermore, operating system 1230 can provide runtime environments, such as the Java runtime environment or the .NET framework, for applications 1232. Runtime environments are consistent execution environments that allow applications 1232 to run on any operating system that includes the runtime environment. Similarly, operating system 1230 can support containers, and applications 1232 can be in the form of containers, which are lightweight, standalone, executable packages of software that include, e.g., code, runtime, system tools, system libraries and settings for an application.

Further, computer 1202 can be enabled with a security module, such as a trusted processing module (TPM). For instance, with a TPM, boot components hash next in time boot components, and wait for a match of results to secured values, before loading a next boot component. This process can take place at any layer in the code execution stack of computer 1202, e.g., applied at the application execution level or at the OS kernel level, thereby enabling security at any level of code execution.

A user can enter commands and information into the computer 1202 through one or more wired/wireless input devices, e.g., a keyboard 1238, a touch screen 1240, and a pointing device, such as a mouse 1242. Other input devices (not shown) can include a microphone, an infrared (IR) remote control, a radio frequency (RF) remote control, or other remote control, a joystick, a virtual reality controller or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1204 through an input device interface 1244 that can be coupled to the system bus 1208, but can be connected by other interfaces, such as a parallel port, an IEEE 1394 serial port, a game port, a USB port, an IR interface, a BLUETOOTH® interface, etc.

A monitor 1246 or other type of display device can be also connected to the system bus 1208 via an interface, such as a video adapter 1248. In addition to the monitor 1246, a computer typically includes other peripheral output devices (not shown), such as speakers, printers, etc.

The computer 1202 can operate in a networked environment using logical connections via wired or wireless communications to one or more remote computers, such as a remote computer(s) 1250. The remote computer(s) 1250 can be a workstation, a server computer, a router, a personal computer, portable computer, microprocessor-based entertainment appliance, a peer device or other common network node, and typically includes many or all of the elements described relative to the computer 1202, although, for purposes of brevity, only a memory/storage device 1252 is illustrated. The logical connections depicted include wired/wireless connectivity to a local area network (LAN) 1254 or larger networks, e.g., a wide area network (WAN) 1256. Such LAN and WAN networking environments are commonplace in offices and companies, and facilitate enterprise-wide computer networks, such as intranets, all of which can connect to a global communications network, e.g., the Internet.

When used in a LAN networking environment, the computer 1202 can be connected to the local network 1254 through a wired or wireless communication network interface or adapter 1258. The adapter 1258 can facilitate wired or wireless communication to the LAN 1254, which can also include a wireless access point (AP) disposed thereon for communicating with the adapter 1258 in a wireless mode.

When used in a WAN networking environment, the computer 1202 can include a modem 1260 or can be connected to a communications server on the WAN 1256 via other means for establishing communications over the WAN 1256, such as by way of the Internet. The modem 1260, which can be internal or external and a wired or wireless device, can be connected to the system bus 1208 via the input device interface 1244. In a networked environment, program modules depicted relative to the computer 1202 or portions thereof, can be stored in the remote memory/storage device 1252. It will be appreciated that the network connections shown are examples and other means of establishing a communications link between the computers can be used.

When used in either a LAN or WAN networking environment, the computer 1202 can access cloud storage systems or other network-based storage systems in addition to, or in place of, external storage devices 1216 as described above, such as but not limited to a network virtual machine providing one or more aspects of storage or processing of information. Generally, a connection between the computer 1202 and a cloud storage system can be established over a LAN 1254 or WAN 1256 e.g., by the adapter 1258 or modem 1260, respectively. Upon connecting the computer 1202 to an associated cloud storage system, the external storage interface 1226 can, with the aid of the adapter 1258 or modem 1260, manage storage provided by the cloud storage system as it would other types of external storage. For instance, the external storage interface 1226 can be configured to provide access to cloud storage sources as if those sources were physically connected to the computer 1202.

The computer 1202 can be operable to communicate with any wireless devices or entities operatively disposed in wireless communication, e.g., a printer, scanner, desktop or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

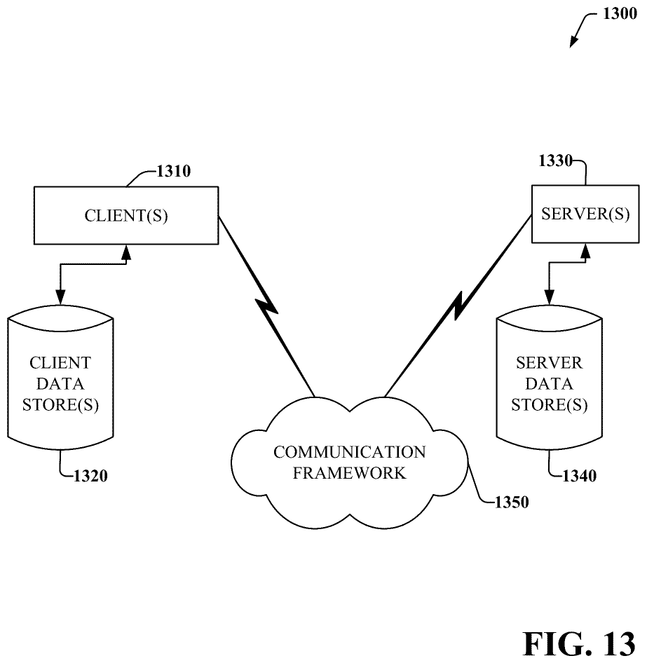

FIG. 13 is a schematic block diagram of a sample computing environment 1300 with which the disclosed subject matter can interact. The sample computing environment 1300 includes one or more client(s) 1310. The client(s) 1310 can be hardware or software (e.g., threads, processes, computing devices). The sample computing environment 1300 also includes one or more server(s) 1330. The server(s) 1330 can also be hardware or software (e.g., threads, processes, computing devices). The servers 1330 can house threads to perform transformations by employing one or more embodiments as described herein, for example. One possible communication between a client 1310 and a server 1330 can be in the form of a data packet adapted to be transmitted between two or more computer processes. The sample computing environment 1300 includes a communication framework 1350 that can be employed to facilitate communications between the client(s) 1310 and the server(s) 1330. The client(s) 1310 are operably connected to one or more client data store(s) 1320 that can be employed to store information local to the client(s) 1310. Similarly, the server(s) 1330 are operably connected to one or more server data store(s) 1340 that can be employed to store information local to the servers 1330.

Various embodiments may be a system, a method, an apparatus or a computer program product at any possible technical detail level of integration. The computer program product can include a computer readable storage medium (or media) having computer readable program instructions thereon for causing a processor to carry out aspects of various embodiments. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network or a wireless network. The network can comprise copper transmission cables, optical transmission fibers, wireless transmission, routers, firewalls, switches, gateway computers or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of various embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform various aspects.

Various aspects are described herein with reference to flowchart illustrations or block diagrams of methods, apparatus (systems), and computer program products according to various embodiments. It will be understood that each block of the flowchart illustrations or block diagrams, and combinations of blocks in the flowchart illustrations or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks. These computer readable program instructions can also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart or block diagram block or blocks.

The flowcharts and block diagrams in the Figures illustrate the architecture, functionality, and operation of possible implementations of systems, methods, and computer program products according to various embodiments. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer or computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that various aspects can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, or a computer. By way of illustration, both an application running on a server and the server can be a component. One or more components can reside within a process or thread of execution and a component can be localized on one computer or distributed between two or more computers. In another example, respective components can execute from various computer readable media having various data structures stored thereon. The components can communicate via local or remote processes such as in accordance with a signal having one or more data packets (e.g., data from one component interacting with another component in a local system, distributed system, or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. As used herein, the term “and/or” is intended to have the same meaning as “or.” Moreover, articles “a” and “an” as used in the subject specification and annexed drawings should generally be construed to mean “one or more” unless specified otherwise or clear from context to be directed to a singular form. As used herein, the terms “example” or “exemplary” are utilized to mean serving as an example, instance, or illustration. For the avoidance of doubt, the subject matter disclosed herein is not limited by such examples. In addition, any aspect or design described herein as an “example” or “exemplary” is not necessarily to be construed as preferred or advantageous over other aspects or designs, nor is it meant to preclude equivalent exemplary structures and techniques known to those of ordinary skill in the art.

The herein disclosure describes non-limiting examples. For ease of description or explanation, various portions of the herein disclosure utilize the term “each,” “every,” or “all” when discussing various examples. Such usages of the term “each,” “every,” or “all” are non-limiting. In other words, when the herein disclosure provides a description that is applied to “each,” “every,” or “all” of some particular object or component, it should be understood that this is a non-limiting example, and it should be further understood that, in various other examples, it can be the case that such description applies to fewer than “each,” “every,” or “all” of that particular object or component.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a processor that executes computer-executable components stored in memory, wherein the computer-executable components comprise:
a reconstruction component that reconstructs one or more material decomposed CT images by balancing a forward model agent and a prior model agent, wherein the forward model agent represents a conditional distribution of observed data given an unknown CT image, wherein the prior model agent represents an assumed prior distribution, and wherein the observed data includes measurements from a photon counting detector.

2. The system of claim 1, further comprising:
a definition component that defines the forward model agent and the prior model agent in a path length sinogram domain.

3. The system of claim 1, further comprising:
a generation component that generates the forward model agent and the prior model agent, wherein the forward model agent is designed to reduce a negative log likelihood of the measurements, and wherein the prior model agent is designed to generate a smooth reconstructed CT image.

4. The system of claim 1, wherein the forward model agent is further designed to estimate path lengths of basis materials, wherein the forward model agent reduces a negative log likelihood of a Poisson photo counting model, and wherein a DRF in the forward model agent is approximated by a low-degree polynomial or a member of a low-dimensional set of functions.

5. The system of claim 1, wherein the prior model agent is designed using machine learning techniques.

6. The system of claim 3, wherein generating the forward model agent and prior model agent decomposes a CT image reconstruction problem into separate algorithms that can be tuned individually.

7. A computer-implemented method, comprising:
reconstructing, by a device operatively coupled to a processor, one or more material decomposed CT images by balancing a forward model agent and a prior model agent, wherein the forward model agent represents a conditional distribution of observed data given an unknown CT image, wherein the prior model agent represents an assumed prior distribution, and wherein the observed data includes measurements from a photon counting detector.

8. The computer-implemented method of claim 7, further comprising:
defining, by the device, the forward model agent and the prior model agent in a path length sinogram domain.

9. The computer-implemented method of claim 7, further comprising:
generating, by the device, the forward model agent and the prior model agent, wherein the forward model agent is designed to reduce a negative log likelihood of the measurements, and wherein the prior model agent is designed to generate a smooth reconstructed CT image.

10. The computer-implemented method of claim 7, wherein the forward model agent is further designed to estimate path lengths of basis materials, and wherein the forward model agent reduces a negative log likelihood of a Poisson photo counting model.

11. The computer-implemented method of claim 7, wherein the prior model agent is designed using machine learning techniques.

12. The computer-implemented method of claim 7, wherein a DRF in the forward model agent is approximated by a low-degree polynomial or a member of a low-dimensional set of functions.

13. A computer program product for modular material decomposition from energy resolving photon counting data, the computer program product comprising a non-transitory computer readable memory having program instructions embodied therewith, the program instructions executable by a processor to cause the processor to:
reconstruct one or more material decomposed CT images by balancing a forward model agent and a prior model agent, wherein the forward model agent represents a conditional distribution of observed data given an unknown CT image, wherein the prior model agent represents an assumed prior distribution, and wherein the observed data includes measurements from a photon counting detector.

14. The computer program product of claim 13, wherein the program instructions are further executable by the processor to cause the processor to:
define the forward model agent and the prior model agent in a path length sinogram domain.

15. The computer program product of claim 13, wherein the program instructions are further executable by the processor to cause the processor to:
generate the forward model agent and the prior model agent, wherein the forward model agent is designed to reduce a negative log likelihood of the measurements, and wherein the prior model agent is designed to generate a smooth reconstructed CT image.

16. The computer program product of claim 13, wherein the forward model agent is further designed to estimate path lengths of basis materials, and wherein the forward model agent reduces a negative log likelihood of a Poisson photo counting model.

17. The computer program product of claim 13, wherein the prior model agent is designed using machine learning techniques.

18. The computer program product of claim 13, wherein a DRF in the forward model agent is approximated by a low-degree polynomial or a member of a low-dimensional set of functions.

19. A system, comprising:
a processor that executes computer-executable components stored in memory, wherein the computer-executable components comprise:
a reconstruction component that reconstructs one or more material decomposed CT images by iteratively applying a forward model agent and a prior model agent, wherein reconstructing the one or more material decomposed CT images involves using photon counting measurements from a photon counting detector, wherein the forward model agent minimizes a negative log likelihood of the photon counting measurements, and wherein the prior model agent represents a second function that is an assumed prior distribution.

20. A system, comprising:
a processor that executes computer-executable components stored in memory, wherein the computer-executable components comprise:
a reconstruction component that reconstructs one or more material decomposed CT images by iteratively balancing a plurality of functions, wherein reconstructing the one or more material decomposed CT images involves using photon counting measurements from a photon counting detector, wherein at least a first function of the plurality of functions is a forward model agent that minimizes a fit of measured photon counts, and wherein at least a second function of the plurality of functions is a prior model agent that reduces noise in one or more reconstructed material decomposed CT images.
